(19) United States
(12) Patent Application Publication
(10) Pub. No.: US 2006/0057122 A1
(43) Pub. Date: Mar. 16, 2006

(54) Functional assessment, specific enrichment and specific depletion of alloreactive human T cells

(75) Inventors: Krishna Komanduri, Pearland, TX (US); Sergio L.R. Martins, Sao Paulo (BR)

Correspondence Address: VINSON & ELKINS, L.L.P., 1001 FANNIN STREET, 2300 FIRST CITY TOWER, HOUSTON, TX, 77002-6760, US

(73) Assignee: The University of Texas System

(21) Appl. No.: 11/172,388

(22) Filed: Jun. 29, 2005

Related U.S. Application Data
(60) Provisional application No. 60/584,639, filed on Jul. 1, 2004.

(51) Int. Cl. C12N 5/08; A61K 35/14

(52) U.S. Cl. 424/93.7; 435/372

(57) ABSTRACT

Alloreactive immune cell populations, clinical uses thereof and a means for specifically depleting alloreactive immune cells, while sparing other immune cell populations and thereby retaining broad specificity for other immune stimuli are disclosed. The present disclosure relates a means for specifically depleting alloreactive T cells, while sparing other T cell populations with a sorting strategy utilizing phenotypic characteristics to specifically deplete alloreactive T cells while retaining broad specificity for other stimuli, including viral antigens and third party alloantigens, specifically depleted alloreactive T cell populations and clinical uses of such specifically depleted alloreactive T cell populations.

### BACKGROUND OF THE INVENTION

1. Field of the Invention

The invention relates to the fields of immunology and stem cell transplant. More specifically the invention relates to the depletion of alloreactive immune cells, particularly T cells, from populations of allogenic cells.

2. Description of Related Art

Human T cell alloreactivity plays an important role in many disease processes, including the rejection of solid organ grafts and graft-versus-host disease (GVHD) following allogeneic stem cell transplantation. The recognition of alloantigens by human T cells forms the basis for several clinically significant disease processes, including GVHD arising in the setting of stem cell transplantation (SCT) and solid organ allograft rejection. (Bach, et al., 1976; Sprent, et al., 1986) In the setting of human stem cell transplantation, numerous interventions have been attempted to reduce the risk of GVHD. Most of these therapeutic approaches have targeted the number and/or function of T cells transferred from the donor to the recipient. (Ferrara, et al., 1991) In almost all allogeneic stem cell transplants performed today, inhibitors of T cell activation, including cyclosporine A or tacrolimus are administered to SCT recipients. (Ho, et al., 2001; Champlin, et al., 1999) Additional strategies have involved the elimination of T cells within the transferred graft, either by negative depletion of CD3⁺ T cells or by the transfer of positively selected CD34⁺ stem cells resulting in effective reductions of T cell doses. (Reisner, et al., 1981; Prentice, et al., 1982; Filipovich, et al., 1982) In other cases, polyclonal or monoclonal antibodies used to purge the allograft are administered to the recipient shortly after graft infusion, including antithymocyte globulins (Rodt, et al., 1982) or the CD52-specific agent Campath-1H (Waldman, et al., 1984), effectively result in the depletion of graft-derived T cells. The application of one or a combination of these strategies leads to an aggregate reduction in the risk of GVHD after SCT. (Ho, et al., 2001; Cavazzana-Calvo, et al., 2002)

Other investigators have attempted to develop alternate approaches to deplete alloreactive T cells. In one general strategy, cells expressing the CD25 antigen associated with the interleukin-2 receptor are depleted using a toxin-conjugated monoclonal antibody following the stimulation of donor cells with alloantigenic stimulators. (Cavazzana-Calvo, et al., 1990; Montagna, et al., 1999) There is often no discrete level of CD25 expression that is associated with cellular activation. (Stanzani, et al., 2004) Depletion using a toxin-conjugated antibody is not “tunable” in a manner that allows the specific purging of cells above a threshold level of activation marker expression. Thus, there is also a greater potential for nonspecific toxicity toward desired PBMC subsets. Furthermore, it is now well recognized that subsets of CD4⁺ T cells that co-express CD25 have regulatory activity that may suppress the development of GVHD in murine models and preclinical human experiments. (Hoffman, et al., 2002; Matthias Edinger, et al., 2003) Toxin-mediated killing of CD25⁺ T cells would be expected to deplete both alloreactive and regulatory CD25⁺ T cells, potentially offsetting the gains achieved by effective alloantigen-specific depletion. Furthermore, such approaches would also be expected to deplete both CD25⁺ CD8⁺ T cells and natural killer (NK) cells that might mediate graft-versus-tumor responses. Another recently described approach used CFSE-labeling of responder cells to sort CFSE^{low} T cells thought to comprise the alloreactive population. (Godfrey, et al., 2004) While this approach is attractive in its simplicity, it is known that higher concentrations of CFSE may be toxic to T cells in vitro, making the safety of such an approach in humans uncertain.

Unfortunately, the benefits of decreases in morbidity due to GVHD are always. offset by the simultaneous non-specific reduction in the numbers of non-alloreactive T cells, leading to an increased risk of infection and relapse following T cell depletion. (Goldman, et al., 1988; Couriel, et al., 1996) For many SCT candidates, suitable matched donors are not available, precluding the application of this potentially curative treatment modality. Consequently, the vast majority of transplants performed utilize matched sibling or unrelated donors due to our inability to selectively reduce alloreactivity in the setting of mismatched transplantation. The instant invention relates a practical method that allows for the specific depletion or enrichment of alloreactive immune cells, while sparing other immune cell populations.

### BRIEF SUMMARY OF THE INVENTION

The present disclosure is directed to specifically depleted alloreactive immune cell populations, clinical uses thereof and a means for specifically depleting alloreactive immune cells, while sparing other immune cell populations and thereby retaining broad specificity for other immune stimuli. More particularly, the present disclosure relates to a means for specifically depleting alloreactive T cells, while sparing other T cell populations with a sorting strategy utilizing phenotypic characteristics to specifically deplete alloreactive T cells, while retaining broad specificity for other stimuli, including viral antigens and third party alloantigens, specifically depleted alloreactive T cell populations created thereby and clinical uses of such specifically depleted alloreactive T cell populations.

The instant invention arises from the elucidation and characterization of the phenotypic and functional characteristic of T cells responding to allogeneic stimuli. The instant disclosure relates that following the prolonged stimulation of T cells in vitro using either allogeneic stimulator cells or viral antigens, the co-expression of activation markers within the CD4⁺ T cell subset occurs exclusively within a subpopulation of T cells that significantly increased their surface expression of CD4 (“CD4^{hi}” cells). Preferred embodiments of the instant invention involve the isolation and/or depletion of such subpopulations of T cells having significantly increased surface expression of CD4 as well as the isolation and/or depletion of further subsets of this alloreactive T cell subpopulation based upon their expression of a CD4^{hi} phenotype in combination with other readily elucidated phenotypic markers, such as, for example CD38. Preferred embodiments of the instant invention further involve the clinical application of T cell populations selectively depleted of T cells having significantly increased surface expression of CD4 and generally one or more other phenotypic markers in the treatment of transplant related disorders such as GVHD.

In contrast to other previously described approaches to deplete alloreactive T cells, preferred embodiments of the disclosed invention utilize well known sorting techniques based upon easily identified surface markers. The use of two markers in combination greatly enhances the ability to discriminate activated and resting cells by flow cytometry. There is also a historical precedent for the use of sorted or otherwise separated cellular populations in clinical use, and such methods have proven safe in humans. Recent clinical studies have demonstrated that the infusion of relatively modest numbers of polyclonal cytomegalovirus (“CMV”)-specific T cells (i.e., 10⁵/kg) may be sufficient to limit viral reactivation after allogeneic SCT. (Peggs, et al. 2003) Consequently, preferred embodiments of the instant invention utilize standard apheresis products from healthy donors to yield sufficient quantities of alloreactivity-depleted T cells for clinical use, even when it is assumed that the precursor frequencies of alloreactive T cells in the haploidentical or mismatched setting may be significantly lower than those expressly disclosed in the examples herein.

It is expressly contemplated that clinical applications of the disclosed alloreactivity-depleted T cells will be for the reduction or prevention of the rejection of solid organ grafts and GVHD following transplantation, particularly following allogeneic stem cell transplantation. In preferred embodiments of the invention, alloreactive T cells are depleted from a transplant to inhibit rejection of transplanted cells, tissues or organs. Additionally, alloreactive T cells can be depleted from donor bone marrow isolates prior to bone marrow transplantation to inhibit graft versus host disease in a transplant recipient. In alternate embodiments, it is specifically contemplated that autoreactive T cells may be depleted to treat autoimmune disorders and allergen-specific T cells may be depleted to treat or reduce the effect of allergies.

A preferred embodiment of the instant invention involves a method for generating a leukocyte population that is functionally anergic following restimulation with alloantigen, comprising:
a. stimulating a cell population with allogeneic stimulator cells; and
b. depleting T cells from the cell population expressing CD4^{hi} and a second activation marker.
   In alternate aspects of this embodiment, depletion is by flow cytometry. In further aspects of this embodiment the second activation marker is CD38, CD25 or CD58. In a still further aspect of this embodiment, the cell population retains T cells capable of responding to antigens other than those expressed by the allogeneic stimulator cells. In another aspect of this embodiment, the allogeneic stimulator cells comprise dendritic cells.

A further embodiment of the instant invention involves a method of reducing the risk of graft versus host disease in a transplant recipient, comprising:
a. isolating a blood product from a donor;
b. stimulating cells in the blood product with allogeneic stimulator cells;
c. depleting cells from the blood product expressing CD4^{hi} and a second activation marker; and
d. administering the blood product to a recipient.
   In alternate aspects of this embodiment, depletion is by flow cytometry. In further aspects of this embodiment the second activation marker is CD38, CD25 or CD58. In a still further aspect of this embodiment, the cell population retains T cells capable of responding to antigens other than those expressed by the allogeneic stimulator cells. In another aspect of this embodiment, the allogeneic stimulator cells comprise dendritic cells.

A further embodiment of the invention relates to a cellular composition suitable for administration to a transplant recipient, wherein the cellular composition is produced by a process comprising:
a. isolating blood product from a donor;
b. stimulating cells in the blood product with allogeneic stimulator cells;
c. depleting cells from the blood product expressing CD4^{hi} and a second activation marker to form a cellular composition substantially free of cells expressing CD46 and the second activation marker.

In alternate aspects of this embodiment, depletion is by flow cytometry. In further aspects of this embodiment the second activation marker is CD38, CD25 or CD58. In a still further aspect of this embodiment, the cell population retains T cells capable of responding to antigens other than those expressed by the allogeneic stimulator cells. In another aspect of this embodiment, the allogeneic stimulator cells comprise dendritic cells.

Another embodiment of the instant invention relates to a leukocyte population, wherein the leukocyte population is depleted of cells expressing CD4^{hi} and a second activation marker following stimulation with alloantigenic stimulator cells.

In alternate aspects of this embodiment, depletion is by flow cytometry. In further aspects of this embodiment the second activation marker is CD38, CD25 or CD58. In a still further aspect of this embodiment, the cell population retains T cells capable of responding to antigens other than those expressed by the allogeneic stimulator cells. In another aspect of this embodiment, the allogeneic stimulator cells comprise dendritic cells.

Another embodiment of the instant invention relates to a method for treating a patient with a hematopoietic cell cancer comprising administering to the patient purified T cells expressing CD4^{hi} and a second activation marker following stimulation with allogeneic stimulator cells.

In alternate aspects of this embodiment, depletion is by flow cytometry. In further aspects of this embodiment the second activation marker is CD38, CD25 or CD58. In another aspect of this embodiment, the allogeneic stimulator cells comprise dendritic cells.

A further embodiment of the instant invention relates to a method of reducing the risk of graft versus host disease in a transplant recipient, comprising:
a. isolating apheresis product from a donor;
b. stimulating cells in the apheresis product with allogeneic stimulator cells;
c. depleting cells expressing a CD4^{hi}CD38⁺ from the cellular composition by flow cytometry to form a cellular composition substantially free of CD4^{hi} CD38⁺ cells; and
d. administering the cellular composition to a recipient.
   In alternate aspects of this embodiment, the cellular composition is enriched for pathogen specific T cells prior to administration to the recipient. In a still further aspect of this embodiment, the cellular composition is administered to the recipient at a concentration of about 1×10⁴ cells/kg, 5×10⁴ cells/kg, 1×10⁵ cells/kg, 5×10⁵ cells/kg, 1×10⁶ cells/kg, 5×10⁶ cells/kg, 1×10⁷ cells/kg, 5×10⁷ cells/kg, 1×10⁸ cells/kg, 5×10⁸ cells/kg or more. In another aspect of this embodiment, the allogeneic stimulator cells comprise dendritic cells.

A further embodiment of the instant invention relates to a method for reducing the autoimmune T-cells in a patient, comprising:
a. isolating apheresis product from the patient;
b. stimulating cells in the apheresis product with autoantigen;
c. depleting cells expressing a CD4^{hi}CD38⁺ from the cellular composition by flow cytometry to form a cellular composition substantially free of CD4^{hi}CD38⁺ cells; and
d. administering the cellular composition to the patient.

In an alternate embodiment of the instant invention, the adoptive transfer of donor lymphocytes (“donor lymphocyte infusions” or DLI) has been demonstrated to have the potential to induce remissions following relapses of hematological malignancies after allogeneic stem cell transplantation (SCT). Currently, DLI is generally performed by infusing PBMC obtained via apheresis from the stem cell donor of the patient who has relapsed following allogeneic SCT. In some cases, minimal fractionation of the PBMC population (e.g., depletion of CD8+ T cells) has also been done. However, further functional fractionation (e.g., purification of a T cell subpopulation enriched for alloreactivity) has the potential to improve current results using unfractionated DLI. Further preferred embodiments of the instant invention involve the isolation and application of subpopulations of T cells enriched for host-reactive T cells which may then be administered to a patient as a therapy for relapsed malignancy after allogeneic SCT.

In an alternate embodiment of the invention, T cell populations isolated by the disclosed methods may be useful in genetic studies, for example genetic analysis of their phenotype, by for example, gene microarrays.

A further embodiment of the instant invention involves a method for generating a leukocyte population that is enriched following stimulation with a target antigen, comprising:
a. stimulating a cell population with stimulator cells presenting the target antigen; and
b. selecting T cells from the cell population expressing CD4^{hi} and a second activation marker.

In alternate aspects of this embodiment, depletion is by flow cytometry. In further aspects of this embodiment the second activation marker is CD38, CD25 or CD58. In a still further aspect of this embodiment, the cell population retains T cells capable of responding to antigens other than those expressed by the allogeneic stimulator cells. In another aspect of this embodiment, the allogeneic stimulator cells comprise dendritic cells. In another aspect of this embodiment, the target antigen is a tumor antigen, a pathogen antgen or a viral antigen.

A further embodiment of the invention relates to a cellular composition, wherein said cellular composition is produced by a process comprising:
a. isolating blood product from a donor;
b. stimulating cells in said blood product with stimulator cells presenting a target antigen;
c. depleting cells from the blood product expressing CD4^{hi} and a second activation marker to form a cellular composition enriched with cells expressing CD4^{hi} and the second activation marker.

In alternate aspects of this embodiment, depletion is by flow cytometry. In further aspects of this embodiment the second activation marker is CD38, CD25 or CD58. In a still further aspect of this embodiment, the cell population retains T cells capable of responding to antigens other than those expressed by the stimulator cells. In another aspect of this embodiment, the stimulator cells comprise dendritic cells. In another aspect of this embodiment, the target antigen is a tumor antigen, a pathogen antgen or a viral antigen.

### BRIEF DESCRIPTION OF THE SEVERAL VIEWS OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Kinetics of cytokine production in alloreactive human T cells. Cytokine production within donor CD4⁺CD14⁻ and CD4⁻CD14⁻ cells are shown at various intervals following stimulation with pooled allogeneic PBMC. The frequencies of T cells producing intracellular TNFα, IFNγ and IL-2 were assessed by cytokine flow cytometry (CFC). In each graph, frequencies of cells responding to stimulation with allogeneic pooled PBMC (●) and autologous PBMC that were either irradiated (▾) or not irradiated (Δ) are shown. Medians and standard deviations shown were derived from six experiments performed using unique donors and the same allogeneic PBMC pool.

FIG. 2. The use of dendritic cells as allogeneic stimulators. Responder PBMC from a healthy human donor were stimulated in a 1:1 ratio using either irradiated monocyte-derived dendritic cells (left) or irradiated PBMC from the same healthy mismatched donor. In this example, dendritic cell stimulation resulted in increased numbers of alloactivated (CD4^{hi}CD38+) T cells, relative to stimulation using PBMC (2.98% vs. 0.54%). These results are representative of three similar experiments.

FIG. 3. CD4 upregulation occurs following chronic stimulation in vitro. A) Histograms represent CD4 fluorescence intensity following various stimulation periods. A distinct CD4^{hi} population starts to become evident at day six, and is clearly evident at day seven, in data representative of experiments from six unique donors. B) Upregulation of activation markers occurs uniquely on CD4^{hi} T cells. Following seven days of stimulation with an allogeneic PBMC pool, upregulation of ten activation markers (y-axes) was assessed with respect to CD4 fluorescence intensity (x-axis). In each case, activation marker co-expression was restricted within the CD4⁺ T cell subset to the CD4^{hi} population. Data from one donor shown are representative of experiments from eight unique donors stimulated in the same fashion. C) Proliferation of alloreactive T cells is restricted to the CD4^{hi} subset. A decrease in fluorescence intensity of CFSE, a dye used to label responder cells, is seen within the CD4⁺ T cell population only in the CD4^{hi} subset following seven days of stimulation. D) Within the CD4+ T cell population, CFSE^{low} cells are confined to the alloreactive CD4^{hi}CD38⁺ T cell subset following allogeneic stimulation. The median number of divisions, calculated by the change in CFSE fluorescence intensity in the two peaks illustrated at right, is 4.1±0.5, based on experiments from five unique donors. Assuming that non-dividing cells did not die during the stimulation period, we estimate that the original precursor frequency of alloreactive cells was 6.9%±1.5% of the original CD4+ T cell population.

FIG. 4. Specific depletion of alloreactive and/or CMV-specific CD4+ T cells. A) The baseline frequencies of responder CD4+ T cells from a healthy CMV-seropositive donor responding to seven days of stimulation with autologous PBMC, pooled allogeneic PBMC and CMV antigens are shown. In each case, the TNFα+ frequency within the CD4⁺ T cell population is shown (upper right quadrants). B) Specific depletion of CD4^{hi}CD38⁺ T cells following allogeneic or CMV stimulation. Following primary stimulation with either pooled allogeneic PBMC (top) or CMV lysates (bottom), high-speed sorting was used to deplete the cells contained within the CD4^{hi}CD38⁺ population (black squares). Residual cells, including CD4^{low}CD38⁻ cells and all CD8⁺ T cells, were then re-stimulated with autologous PBMC, pooled allogeneic PBMC and CMV antigens. Following primary allogeneic stimulation and depletion of CD4^{hi}CD38⁺ cells, secondary stimulation induced a CMV-specific CD4⁺ T cell response slightly higher than that at baseline (2.99% vs. 1.44%), while secondary stimulation with the allogeneic PBMC pool was reduced to a level similar to that following control autologous stimulation (0.22% vs. 0.23%). Conversely, depletion of CD4^{hi}CD38⁺ cells following primary CMV stimulation resulted in preserved secondary CD4⁺ T cell responses to allogeneic stimulation (5.49%) but not CMV stimulation (0.17% vs. 0.16% following autologous stimulation). As shown at right, depletion of the CD4^{hi}CD38⁺ T cell subset following either allogeneic or CMV stimulation also resulted in the loss of a detectable CD8⁺ T cell response following secondary re-stimulation with the same antigen (0.23% for allogeneic and 0.19% after CMV stimulation), similar to that in control samples (data not shown). Data shown are representative of five depletion experiments following allogeneic stimulation and four experiments depleting CMV-specific T cells from unique donors. C) Aggregate results from nine separate experiments are shown. (five from unique donors depleting allogeneic CD4^{hi}CD38+ responders, four depleting CMV-specific CD4^{hi}CD38⁺ responders). Displayed are the medians and standard deviations of TNFα-producing cells within CD4⁺ and CD8⁺ T cell subsets prior to either allogeneic depletion (top left panels) or CMV depletion (bottom left panels). Following depletion of either allogeneic or CMV specific CD4^{hi}CD38⁺ T cells, responses to the primary stimulus in both CD4⁺ and CD8⁺ T cell compartments during secondary stimulation were reduced to a level indistinguishable to that seen following control autologous stimulation (right panels).

FIG. 5. T cell receptor spectratyping reveals the presence of a diverse T cell receptor (TCR) repertoire following depletion of alloreactive or CMV-specific T cells. A) Molecular diversity of CD4^{hi}CD38⁺ T cells. Purified CD4^{hi}CD38⁺ T cells obtained following CMV stimulation demonstrated more skewing from a diverse Gaussian pattern relative to the CD4^{hi}CD38⁺ T cell subset obtained after allogeneic stimulation. B) Diverse repertoire of residual CD4^{int}CD38⁻ T cells following depletion of reactive CD4^{hi}CD38⁺ T cells. The TCR repertoire of CD4^{int}CD38⁻ T cells, obtained after either CMV or allogeneic stimulation, was extremely diverse as evidenced by a normal Gaussian TCR spectratype. In each example, representative TCR Vβ subsets from 12 of 23 assessed TCR Vβ subsets. Results are from a single donor and representative of two similar experiments.

FIG. 6. Third-party alloreactivity is preserved after depletion of alloreactive cells stimulated with single-donor apheresis products. PBMC from a healthy donor were stimulated with irradiated PBMC obtained from clinically harvested apheresis products from two donors (designated “A” and “B”). Following seven days of stimulation, the CD4^{hi}CD38+ T cell population was depleted using the gating strategy at left. After depletion of alloantigen-specific CD4^{hi}CD38⁺ T cells, repeat stimulation with the original donor resulted in activation of CD4+ T cells similar to that seen following autologous restimulation. However, residual cells remained capable of responding to either a third-party donor (e.g., 3.70% of CD4⁺ T cells still responded functionally to donor B and 1.21% of CD4⁺ T cells responded to CMV stimulation following depletion of cells originally stimulated with donor A, top). Results are representative of two similar experiments from healthy donors.

### DETAILED DESCRIPTION OF THE INVENTION

Alloreactive CD4⁺ T cells may be identified by CD4 upregulation and co-expression of surface activation markers. Sort-based depletion of cells with this phenotype leaves behind a CD4⁺ and CD8⁺ T cell population that is diverse and capable of responding to pathogens, but that is functionally anergic following restimulation with alloantigens.

Flow cytometry may be used to assess the frequencies of CD4⁺ and CD8⁺ T cells responding to alloantigen stimulation, leading to a more complete understanding of the phenotypic composition of the alloreactive T cell repertoire in humans. The use of flow cytometry, particularly CFC, allows for the elucidation of the functional and phenotypic characteristics of individual cells responding to stimulation in the mixed lymphocyte reaction (MLR), leading to the determination that: 1) the bulk of human alloreactivity in the in vitro MLR resides in the CD4⁺ T cell population; 2) the kinetics of alloantigen activation of T cells in the CFC assay are consistent with prior results using lymphocyte proliferation responses to assess alloreactivity; 3) CD4 upregulation is a hallmark of chronic activation following either alloantigen or CMV stimulation; and 4) within the CD4⁺ T cell subset, proliferation and the expression of secondary activation markers are restricted exclusively to CD4⁺ T cells expressing high levels of surface CD4 (CD4^{hi} cells). These observations lead to a strategy to stimulate cells with either viral antigens or pooled alloantigenic stimulator cells and then deplete cells with the activated CD4^{hi}CD38⁺ phenotype. As disclosed herein, the residual PBMC lack the ability to respond to the initial stimulus, but retain the ability to respond to alloantigens or viral antigens, in cells stimulated with CMV or alloantigens, respectively.

Additional experiments established the fact that the alloantigen-depleted T cell population contained a diverse TCR repertoire. Such alloantigen-depleted T cell population may be derived from clinically obtained apheresis products, resulting in the depletion of specific alloreactivity in a one-way MLR with the preservation of other antigen-specific T cell responses and T cells capable of responding to third party alloantigens.

The approach identified would not be expected to deplete CD4⁺CD25⁺ T^{reg} cells that are capable of suppressing alloreactivity in murine models of allogeneic transplantation and in preclinical human studies (Matthias Edinger, et al. 2003; Taylor, et al., 2002) nor NK cells that may also express CD25 and may mediate anti-leukemic responses, providing a potential advantage over approaches utilizing CD25-specific monoclonal antibodies to purge activated T cells expressing the IL-2 receptor. Further, it appears that alloreactivity in the CD8⁺ T cell population is abrogated by the depletion of alloantigen stimulated CD4⁺ T cells. Indeed, while a significant fraction of CD8⁺ T cells also express activation markers following primary allogeneic stimulation, these cells are not capable of effector cytokine production if alloreactive CD4^{hi}CD38⁺ T cells are first depleted prior to re-stimulation with alloantigens. These data are consistent with murine data that suggest that CD4⁺ T cell help is more critical to the functional reactivation of CD8⁺ T cells than for the development of a primary response. (Janssen, et al., 2003)

The alloantigen-depleted T cell population of the instant invention retains the ability to respond to third party alloantigens while lacking alloreactivity to the recipient. The flow cytometry methods utilized to initially isolate this alloreactive T cells may also be used to deplete this phenotype from cell population for use in, for example, transplant applications. In clinical applications, this population would thus maintain its ability to recognize otherwise foreign antigens in the recipient without attacking the host, thereby causing GVHD or other related ailments. In a preferred embodiment of the invention, tissue, such as blood product is isolated from a donor. Immune cells are isolated from the donor tissue and stimulated with allogeneic stimulator cells as described above. The immune cell population is then depleted of T cells expressing high levels of the CD4 marker and also a second marker of T cell activation such as CD38. The resultant population, depleted of the alloreactive T cells expressing CD4^{hi}CD38, are then dispersed in a pharmaceutically acceptable carrier and administered to a recipient. Because of the depletion of alloreactive T cells, the incidence of adverse reactions in the host to the transplant, i.e. GVHD, should be reduced, curtailed or otherwise eliminated.

In a particularly preferred embodiment of the invention, apheresis product is obtained from a suitable donor and PBMC are isolated therefrom. The PBMC are then stimulated with alloantigenic stimulator cells isolated from the recipient patient. Following an incubation period, the PBMC are labeled with fluorescently labled anti-CD4 and fluorescently labeled anti CD38 antibodies. The cells are then sorted by flow cytometry, with T cells expressing CD4^{hi}CD38 depleted from the PBMC. The remaining population is then placed in a suitable pharmaceutical carrier for administration to the recipient patient.

CFC-based assessment of alloreactivity may further have independent clinical value as a marker of the risk for GVHD, and as a measure of the success of clinical strategies to eliminate GVHD-mediating T cells in vitro and in human subjects. In the setting of immunologically matched allogeneic transplantation, as is performed using sibling donors, the risk of GVHD is incompletely predicted by the degree of matching. For example, approximately 40% of recipients of matched sibling transplants will experience GVHD. CFC-based assessment of alloreactivity may prove to have predictive value in determining the subset of recipients that are most likely to experience GVHD. Donor PBMC would be stimulated with recipient cells that have been irradiated. At a defined interval following co-culture of responder (i.e., donor-derived cells) and stimulator cells (i.e., from the prospective transplant recipient) the fraction of cells from the donor in the CD4+ and/or CD8+ T cell compartment that upregulates surface activation markers and produces effector cytokines (e.g., TNFα, IFNγ or IL-2) could be assessed. It would be expected that higher frequencies of responder cells that become functionally activated in this manner would be found in donor-recipient pairs where clinical GVHD is most likely to occur after allogeneic stem cell transplantation. Similarly, such a method might be used to select amongst potential mismatched family donors or matched or mismatched unrelated donors when more than one potential donor exists. Transplantation using a donor whose cells are less frequently activated by recipient cells (using the CFC alloreactivity assay as an endpoint) would be predicted to result in less clinical GVHD; thus, morbidity and mortality following allogeneic stem cell transplantation might be improved.

Phenotypic Markers

The instant invention contemplates the administration of cellular populations depleted of alloreactive immune cells, wherein the alloreactive immune cells are selected based upon phenotypic characteristics indicating that they were activated in response to alloantigen stimulation. The instant invention relates that, in T cells, the phenotype of alloreactive T cells may be characterized by the surface expression of high levels of surface CD4. In preferred embodiments of the invention, the alloreactive T cells phenotype is further characterized by the surface expression of a second surface marker, in a particularly preferred embodiment, by the surface expression of CD38. In alternate embodiments of the invention this second surface marker may include, but is not limited to indicators of immune cell activation, preferably T cell activation, including, for example: CD2, CD3, CD6, CD7, CD9, CD25, CD25, CD26, CD28, CD45RO, CD49a, CD56, CD58, CD69, CD70, CD71, CD74, CD80, CD82, CD86, CD87, CD94, CD95, CD96, CD97, CD100, CD101, CD109, CD121a, CD122, CD124, CD126, CD127, CDw128a, CD132, CD134, CDw137, CD152, CD153, CD154, CD157, CD160, CD161, CD162, CD166, CD173, CD174, CD178, CD183, CD200, CDw210, CD212, CD213a1, CD223, CD227 and CD229. It is expressly contemplated that, as with the CD4 receptor, the expression level of the various second marker may be relevant in the determination that the marker is indicative of an alloreactive phenotype.

Antibodies to CD4, CD38 and most other surface markers contemplated for use in this invention are readily available from a number of vendors that would be well known to one of skill in the art. In general, these antibodies may be obtained fluorescently labeled with a selectable marker such as FITC or other fluorescent marker. Exemplary sources for fluorescently labeled antibodies include: Molecular Probes, USB, Insight Biotechnology, KPL, BD Biosciences, Pharmingen and Beckman Coulter. In the event that a fluorescently labeled antibody cannot be obtained from commercial sources, means for the fluorescent labeling of antibodies are well known within the art. See Immunochemical Methods In Cell And Molecular Biology (1987); Handbook Of Experimental Immunology, Volumes I-IV (1986), herein incorporated by reference.

Sources of Cells for Clinical Applications

The instant invention contemplates the administration of cellular populations depleted of alloreactive immune cells, primarily alloreactive T cells to a patient. In the clinical setting this would first involve the isolation of transplant materials from a donor. Particular embodiments of the instant invention contemplate that cells for such administration will be derived from the aphresis products of donors. Further embodiments contemplate that donor cells will be obtained from solid organ grafts. While these donors may be HLA matched or haploididentical, it is generally contemplated that the transplant will utilize matched sibling or unrelated donors.

The number of donor cells depleted of alloreactive immune cells administered to a recipient can be increased by either increasing the number of cells provided in a particular administration or by providing repeated administrations of donor cells. In preferred embodiments of the invention, donor cells depleted of alloreactive immune cells are delivered repeatedly to a patient to facilitate and promote engrafiment of the donor cells.

To achieve the requisite numbers of cells for administration ex vivo expansion or amplification of donor cell populations may be required. See U.S. Pat. No. 6,558,662, herein expressly incorporated by reference. Ex vivo expansion is reviewed in Emerson, 1996, Blood 87:3082, hereby incorporated by reference. Methods of ex vivo expansion are described in more detail in Petzer et al., 1996; Zundstra et al., 1994 and WO 95 11692 Davis et al., all of which are hereby incorporated by reference. Although it is generally contemplated that the instant invention will utilize cells derived from apheresis products, sources of cells for transplant include, but are not limited to bone marrow cells, mobilized peripheral blood cells, and when available cord blood cells. It is expressly contemplated that ex vivo expansion of cell populations may occur prior to or subsequent to the depletion of the alloreactive immune cells from the cell population.

Peripheral blood cells may be obtained from the donor, for example, by standard phlebotomy or apheresis techniques. Phlebotomy is performed by placing a hollow needle into a vein and withdrawing a quantity of whole blood using aspiration or gravity. Apheresis is performed in a similar manner to phlebotomy except the whole blood is anticoagulated and then separated into the constituent formed cellular elements by centrifugation. The mononuclear cell fraction is retained and the remaining plasma and other cellular elements (red blood cells, granulocytes, platelets) are returned to the donor by intravenous infusion.

Dendritic cells may be further isolated by, for example, adherence (see Felzmann, et al., 2003; elutriation (Beckman, Inc.), (see also, Adamson, et al. 2004, Cao, et al. 2000) counterflow elutriation (Gambro, Inc.) (See also, Berger, 2005; Garlie, 2005; Noga, 2001), magnetic bead selection (CliniMACS, Miltenyi Biotec), FACS sorting or other methods well known to one of ordinary skill. Dendritic cells may be isolated from, for example, peripheral blood cells, monocytes, or hematopoietic progenitor cells. (Svane et al., 2003).

In some instances, in order to enhance the number of cells obtained from the donor, methods may be employed to enhance the number of cells present in the apheresis product. In an embodiment of the instant invention, hematopoietic growth factors such as Granulocyte colony stimulating factor (GCSF), granulocyte-monocyte colony stimulating factor (GM-CSF), stem cell factor (SCF) may be administered to the donor prior to apheresis to cytokine mobilize immune cells. Such growth factors may be delivered subcutaneously or intravenously in amounts sufficient to cause movement of hematopoietic stem cells from the bone marrow space into the peripheral circulation. The hematopoietic reconstituting cells can also be derived from fetal or embryonic human tissue that is processed and/or cultured in vitro so as to increase the numbers or purity of primitive hematopoietic elements.

In addition, in preferred embodiments of the invention, cells administered to the recipient may be enriched in vitro from the source population. Methods of expanding source populations are well known in the art, but may include selecting cells that express the CD34 antigen, using combinations of density centrifugation, immuno-magnetic bead purification, affinity chromatography, and fluorescent activated cell sorting, known to those skilled in the art (Baum et al., (1992); Lansdorp et al., (1990); Sato et al., (1991); Smith et al., (1991); Udomsakdi et al., (1991); Udomsakdi et al., (1992)).

Once prepared, the cell populations of the instant invention are typically administered to the recipient in a pharmaceutically acceptable carrier by intravenous infusion. Carriers for these cells can include but are not limited to solutions of phosphate buffered saline (PBS) containing a mixture of salts in physiologic concentrations.

In the context of the invention, the number of cells that should be administered to a specific patient will vary dependent upon the nature of the condition being treated, the condition and age of the patient and the number of cells available. Nevertheless, it is well within the purview of one of ordinary skill to determine the proper dosage of cells that should be administered to a specific patient. It is contemplated that a preferred dosage to be delivered for an allogeneic stem cell transplantation would be approximately 1×10⁸ T cells/kg. This range is nevertheless only exemplary and dosage ranges including: 1×10² cells/kg to 1×10⁹ T cells/kg are expressly contemplated.

Flow Cytometry

Flow cytometry is widely used in the art and is a method well known to one of ordinary skill to sort and quantify specific cell types within a population of cells. In general, flow cytometry is a method for quantitating components or structural features of cells primarily by optical means. Although it makes measurements on one cell at a time, it can process hundreds of thousands of cells in a few seconds. Since different cell types can be distinguished by quantitating structural features, flow cytometry and cell sorting can be used to count and sort cells of different phenotypes in a mixture.

A flow cytometric analysis involves two basic steps: 1) labeling selected cell types with one or more labeled markers, and 2) determining the number of labeled cells relative to the total number of cells in the population. In a generalized approach, cells labeled with a fluorescent tag are suspended in a stream which is passed through an illumination and light detection system. This stream is passed dropwise through a light source, generally a laser beam that excites fluorescence of labeled cells, with the cells traversing the illumination spot one by one. As the cells pass through the beam, the fluorescent signals and intensities are digitally measured.

For sorting purposes, the drops in which individual cells are suspended are charged. An electrostatic field in the sorter deflects drops into collecting tubes based upon the signal produced by the cells. Utilizing this methodology, high speed cell sorters are capable of identifying and selecting fluorescently labeled cells at a rate of ˜25,000 per second or 100,000,000 per hour.

The primary method of labeling cell types is by binding labeled antibodies to markers expressed by the specific cell type. The antibodies are either directly labeled with a fluorescent compound or indirectly labeled using, for example, a fluorescent-labeled second antibody which recognizes the first antibody.

Commercially available equipment, e.g., equipment manufactured by such companies as Coulter Electronics, Hialeah, Fla., Becton-Dickinson, Mountain View, Calif., Cytopeia, Seattle, Wash. and Cytomation, Carpenteria, Calif., is widely available for performing the cell sorting and counting step of the analysis. A general discussion of the apparatus of method for flow cytometry is detailed in U.S. Pat. No. 3,826,364, herein incorporated by reference.

Preferred embodiments of the instant invention utilize flow cytometry and cell sorting technology to segregate alloreactive cells from a cellular population. In particular embodiments, flow cytometry and cell sorting are used to specifically enhance or specifically deplete alloreactive T cells from immune cell populations. Although it is generally contemplated that flow cytometry will be utilized in the context of the instant invention to isolate and or deplete alloreactive immune cells, other methods of isolating cells well known to those of ordinary skill in the art, such as, for example, magnetic beads, MACS technology (Miltenyi Biotec, Bergisch Gladbach, Germany) and/or Photodynamic cell purging methods (Goggins, et al. 2003) are also expressly contemplated to be means that may be utilized to segregate alloreactive cells and practice this invention.

Clinical Applications

The instant invention is contemplated to have clinical applications to diseases in which alloreactive immune responses are possible. Preferred embodiments relate to the clinical administration of transplants depleted of alloreactive immune cells. Diseases that are specifically contemplated to be targeted by the disclosed approaches include: Acute myeloid leukemia, chronic myeloid leukemia, non-Hodgkin's lymphoma, Hodgkin's disease, acute lymphoid leukemia, chronic lymphocytic leukemia, multiple myeloma, Waldenstrom's macroglobulinemia, myelodysplasia, myelofibrosis, aplastic anemia, renal cell carcinoma, hemoglobinopathies (including thallasemias and sickle cell diseases). Other diseases where allogeneic stem cell transplantation may have clinical relevancy to include: breast carcinoma, ovarian carcinoma, autoimmune diseases (including scleroderma, Sjogren's syndrome and systemic lupus erythematosus, as well as common neoplasms like lung cancer, colon cancer, and a broader range of hematological, immunodeficiency (i.e. SCID) and autoimmune diseases.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

To study the human T cell subsets responding to allogeneic stimuli, PBMC from a group of ten HLA-disparate individuals were pooled to provide a stimulus for responder T cells. To prevent proliferation from pooled stimulator PBMC, these cells were irradiated prior to incubation with responder cells. Responder PBMC were isolated from healthy donors and co-cultured with pooled allogeneic stimulator cells for varying periods. Following stimulation, the T cells were washed and brefeldin A, an inhibitor of intracellular transport, was added to enable accumulation of effector cytokines in the cytoplasm. Responder T cells were then examined for the simultaneous expression of phenotypic markers associated with T cell maturation or activation, and the production of effector cytokines by CFC.

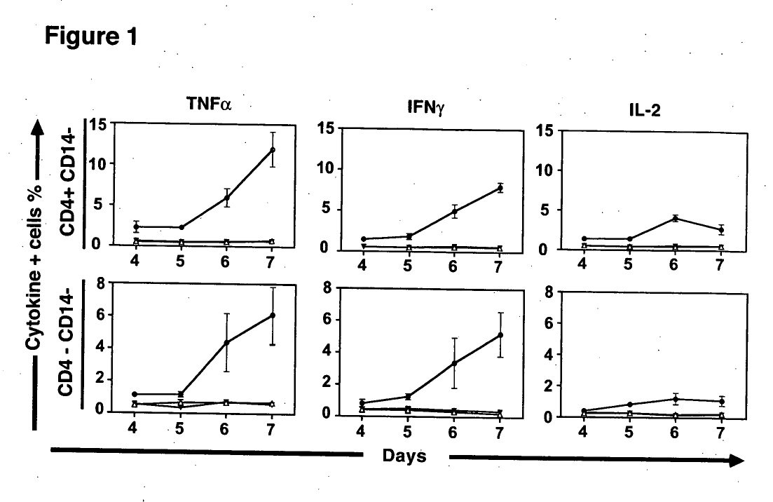

Stimulator cells were labeled with PKH-2 to determine how long irradiated allogeneic stimulators persisted in mixed lymphocyte reactions. PKH-2⁺ stimulator cells were present in declining numbers over the first 24-48 hours of stimulation but were undetectable thereafter. These data suggest that indirect allorecognition is the primary mechanism operative in mixed lymphocyte reactions, following an initial period where both direct and indirect allorecognition may occur. Following stimulation with pooled allogeneic stimulator cells the production of effector cytokines, including tumor necrosis factor-α (TNFα), was induced in responder cells (6.85% of CD4⁺ T cells). In contrast, significant TNFα production in CD4⁺ T cells stimulated with autologous cells was observed, regardless of whether autologous stimulators were irradiated (≦0.32% of CD4⁺ T cells). Consistent with prior observations derived from lymphocyte proliferation assays, cytokine production was evident following approximately five days of allogeneic stimulation. The kinetics of effector cytokine production were similar, irrespective of whether the production of interleukin-2 (IL-2), interferon-γ (IFNγ) or TNFα was assessed (FIG. 1). Cytokine production was seen exclusively within T cells, as assessed by forward and side scatter and by the lack of CD14 expression characteristic of human monocytes. The majority of cytokine production was seen in CD4⁺CD14⁻ T cells, though a smaller proportion of CD8⁺ T cells (demarcated by the CD4⁻CD14⁻ population within the T cell gate) also produced effector cytokines. Results shown are representative of several similar experiments.

Preparation of responder and stimulator cells. PBMC were isolated by ficoll density gradient sedimentation from heparinized whole blood of normal healthy volunteer donors who were CMV seropositive to facilitate characterization of viral-specific T cell responses. PBMC from ten unique donors were irradiated at 25 Gy, pooled and cryopreserved for subsequent use in allogeneic stimulation experiments. For control stimulations, autologous PBMC obtained from healthy donors were similarly irradiated and cryopreserved, prior to co-incubation with autologous responder cells.

Cytokine flow cytometry assay. For initial experiments establishing the kinetics of cytokine production and activation marker co-expression in the cytokine flow cytometry assay, 106 freshly isolated responder PBMC were co-incubated with either CMV lysates (BioWhittaker, Walkersville, Md.) or with an equal number of thawed autologous or pooled allogeneic PBMC in 24-well tissue culture plates in 2 ml of media (RPMI1640, GIBCO Life Technologies, Grand Island, N.Y.) supplemented with 10% human AB serum, L-glutamine, penicillin, and streptomycin (Sigma, St. Louis, Mo.). Stimulator cells were labeled with the membrane dye PKH-2 (Sigma) to enable identification of activation within responder cells. Following various stimulation periods, cells were washed and brefeldin A (Sigma) was added to enable accumulation of effector cytokines in the cytoplasm. Cells were then permeabilized with FACSPerm Solution II (BD Biosciences, San Jose, Calif.) and examined responder T cells for the simultaneous expression of phenotypic markers associated with T cell activation, and the intracellular expression of effector cytokines.

Assessment of T cell activation by flow cytometry. FACS analyses were performed using FITC-, PE-, PerCP-, and APC-conjugated monoclonal antibodies (MAb) specific for human CD4, CD8, CD14, CD38, CD25, CD69, HLA-DR, CD71, CD58, CD122, CD152, CD103, CD134, anti-IFNγ, anti-TNFα and anti-IL-2 (BD Biosciences). After staining, cells were washed, resuspended in PBS with 1% paraformaldehyde, and analyzed by four-color flow cytometry on a FACSCalibur cytometer using Cell Quest software (both BD Biosciences), and FlowJo software (Treestar, San Carlos, Calif.). For most analyses, at least 100,000 total events were analyzed, with sequential gating of PBMC in a lymphocyte region (by scatter), on CD14-negative events to exclude monocytes, and on antigen-specific T cells (by assessing the frequencies of CD4⁺ or CD8⁺ T cells staining positive for intracellular TNFα, IL-2 or IFNγ). Nonspecific activation was assessed by incubation of paired samples that were either unstimulated or stimulated with autologous control cells. The observed frequency of cytokine-producing T cells following autologous and CMV control stimulation was generally <0.2%.

CFSE proliferation assay. To perform quantitation of proliferation in responder cells, responder cells were labeled with CFSE (Molecular Probes, Eugene, Oreg.), a highly fluorescent dye that is transferred to daughter cells, resulting in a linear decrease in fluorescence. PBMC were isolated and labeled with 0.6 μM CFSE for 10 minutes at RT. The reaction was then quenched with AB serum, and then washed two times with RPMI 1640 supplemented with 10% AB serum. The starting frequency of alloreactive T cells was estimated based on the median of fluorescence intensity (MFI), and the percentage of proliferating cells after seven days MLR culture. The precursor frequency of the starting population was calculated based on the following formula, where a=MFI(CD4hi population), b=MFI(CD4^{int} population), c=frequency of CD4^{hi}CD38⁺ T cells following alloantigen stimulation (in %), and n=cell divisions in the alloreactive T cell population (log2(a/b)): Precursor frequency=[100c/2n]/[(100c/2n)+(1-100c)].

### EXAMPLE 2

Stimulation may be accomplished using dendritic cells or PBMC. Initial experiments were performed as noted above using stimulation with peripheral blood mononuclear cells (PBMC) derived from individuals or pools of allogeneic donors. In some cases it may prove advantageous to stimulate responder cells using alternate subsets of cells derived from non HLA-identical individuals. Monocyte-derived dendritic cells (DC) are known to be robust stimulators of allogeneic T cell responses. For this reason, the ability of allogeneic DC to serve as stimulators in the activation phase of the allodepletion process was evaluated.

Initially, monocytes were derived from PBMC using adherence to tissue culture plastic as a means of enriching the monocyte fraction. This results in significant enrichment of CD14+ monocytes from which DC may be derived. However, alternate approaches to isolate monocytes exist and are also envisioned as embodiments of this approach. Such methods of enrichment of monocytes for DC preparation include counterflow elutriation (Gambro, Inc.), CD14+ cell selection using magnetic bead selection (CliniMACS, Miltenyi Biotec) (Padley et al. (2001); Dzionek et al. (2000)) or FACS sorting. Each yields enriched populations of CD14+ monocytes (of varying purity) that are suitable for subsequent preparation of DC.

Using the adherence approach, non-adherent cells are washed following a 3 hr adherence interval. Media (AIM V supplemented with 10% human AB serum and penicillin/streptomycin) is added containing 1000 IU/ml GM-CSF (Leukine®, sargramostim, Berlex, Seattle) and 500 IU/ml interleukin-4 (IL-4). The cells are then incubated for 5 days to allow differentiation into monocyte-derived immature DC. Cells are then washed and new media containing cytokines that facilitate DC maturation is then added for an additional 48 hrs. Such cytokines (or combinations of cytokines and other reagents) may include TNF-α alone (100 IU/ml), or TNFα in combination with IL-1β, IL-6 and prostaglandin E₂ (PGE₂). Following maturation, DC alter their morphology, upregulate surface markers of maturation (e.g., CD83), and decrease their adherence to culture flasks.

Following maturation of DC, stimulation is accomplished as previously described for PBMC-based allogeneic activation. Briefly, varying ratios of responders to irradiated stimulators are incubated to facilitate activation of responder PBMC as previously described, leading to CD4 upregulation and expression of secondary activation markers on CD4^{hi} T cells. A range of responder PBMC:stimulator DC ratios have been examined (e.g., 1:1 to 20:1) with an expectation that some titration may be necessary to optimize results in individual stimulations. Following stimulation for periods of 6-7 days (at which time CD4 upregulation and secondary activation marker expression appear to be optimal), frequencies of CD4^{hi}CD38+ T cells may be assessed by flow cytometry (to determine the relative frequencies of allogeneic responders) or depleted by FACS sorting to selectively reduce specific alloreactivity against alloantigens expressed by the stimulator DC population.

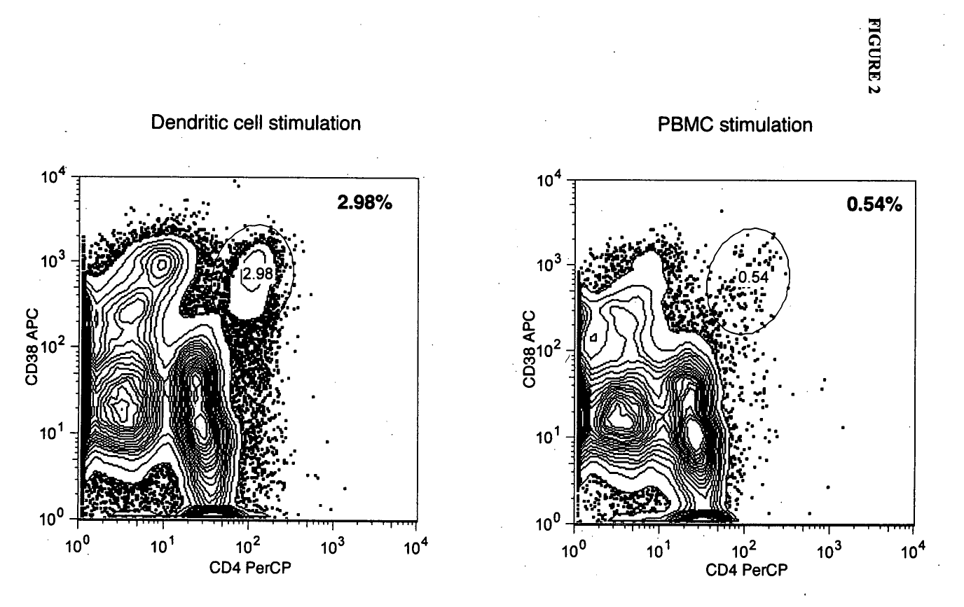

DC stimulation may increase the level of activation in low-frequency responders. In some cases, particularly when the frequency of alloreactive T cells is low in the setting of stimulation with individual cell products, DC-based stimulation may be a useful means of increasing the level of response relative to that seen with PBMC stimulation. An example of this is outlined in FIG. 2. In FIG. 2, paired stimulations were accomplished using stimulator cells derived from the same individual donor. Responder cells from an allogeneic healthy donor were stimulated in culture for seven days, with the frequencies of alloreactive CD4+ T cells assessed by flow cytometric evaulation of CD4 upregulation and CD38 co-expression as previously described. In the example shown (representative of three similar experiments using individual donor-recipient pairs) the stimulation observed using monocyte-derived DC was superior to that seen using PBMC stimulators (2.98%, left vs. 0.54%, right).

Potential use of PBMC, DC and other allogeneic stimulators. While DC may in some cases prove to be equal to or superior to PBMC stimulators as measured by the frequencies of activated alloreactive T cells, it is also possible that the use of DC may result in the selective depletion of desirable T cell subsets (e.g., cancer-specific T cells) that would not be eliminated following stimulation using PBMC preparations (e.g., due to the expression of unique target antigens on DC that might be shared by recipient cancer cells). Thus, the selective allodepletion approach may involve the use of PBMC as stimulators, the alternate use of DC as stimulators, or combinations of DC and PBMC. Other allogeneic cell types (e.g., non-hematologic allogeneic stimulators) may be used to selectively reduce alloreactivity toward recipient cells that express tissue-restricted target antigens that may serve as targets of alloreactivity in the setting of clinical GVHD (e.g., skin cells, liver cells or intestinal cells).

### EXAMPLE 3

Specific depletion of alloreactive or CMV-specific CD4+ T cells. To deplete antigen-specific CD4⁺ T cells, 10⁷ responder PBMC were stimulated with CMV lysates, an equal number of PBMC from an allogeneic pool, or autologous PBMC. After seven days, the responder cells were harvested, washed and then labeled with anti-CD4PerCP and CD38APC MAb (BD Biosciences). The cells were then sorted using a FACSAria flow cytometer (BD Biosciences), excluding CD4^{hi}CD38⁺ cells. Prior to sort-based allodepletion, cells were resuspended in PBS containing 0.1% AB serum and disodium edetate (5 mM final concentration)(Sigma). Following sorting, cells were washed twice in PBS prior to further functional assessment. For spectratyping studies, both CD4^{hi}CD38⁺ and CD4^{int}CD38⁻ populations were selectively purified. For assessment of residual antigen-specific T cell function, the residual population of cells were rested for 16 h and then re-stimulated (as described above) with either thawed allogeneic or autologous PBMC or CMV lysates. Secondary stimulations were performed for either three or seven days and assessed as described for primary stimulations using the CFC assay.

Preclinical assessment of alloreactive T cell depletion. Cells were obtained from a healthy donor (“donor C”) and stimulated as described above with thawed and irradiated PBMC from clinical apheresis products obtained from two different HLA mismatched donors (A and B). Following seven days of stimulation, CD4^{hi}CD38⁺ alloreactive T cells were depleted by sorting, secondary stimulations was performed and residual autologous, CMV-specific and third-party alloreactivity was then assessed by CFC.

Spectratyping assay: Immediately after sorting, total RNA was extracted using a commercial kit (Tel-Test, Friendswood, Tex.) and cDNA prepared using reverse transcription (Applied Biosystems, Foster City, Calif.). The CDR3 regions in 23 TCR Vβ subsets were then amplified by polymerase chain reaction (PCR) and the resulting PCR products then subjected to capillary electrophoresis and quantitative densitometry to assess the fragment length diversity within each of the TCR Vβ families.

Statistical analyses. Data was collected, analyzed and displayed using Prism software (GraphPad, San Diego, Calif.) and Illustrator software (Adobe, Seattle, Wash.) using Macintosh computers (Apple, Cupertino, Calif.). Nonparametric comparisons were performed using the Mann-Whitney U-test. Intergroup comparisons were performed using a non-paired, two tailed one-way ANOVA test. Values of P<0.05 were considered significant.

### EXAMPLE 4

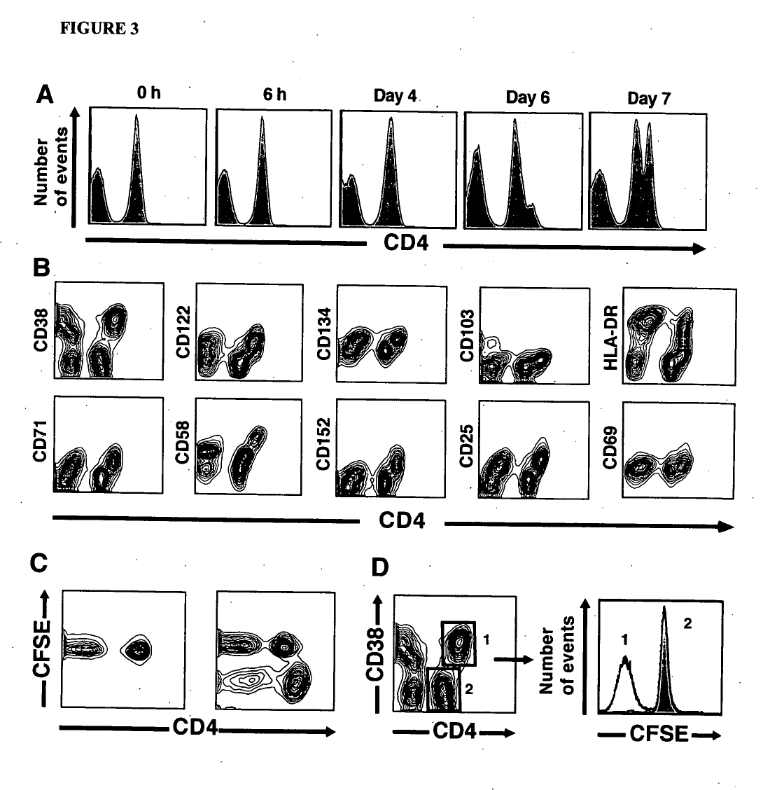

Activation marker expression and proliferation occur exclusively in CD4^{hi} T cells following chronic stimulation. Following short-term stimulation, such as the six-hour period used to stimulate CMV-specific CD4⁺ T cells with viral antigens in CFC assays, the intensity of CD4 expression within CD4⁺ lymphocytes maintains a fairly narrow and Gaussian distribution. In contrast, following chronic stimulation with either CMV antigens or pooled allogeneic stimulator cells, the peak of fluorescence intensity demarcating CD4 expression initially widened, and then became segregated into two distinct populations with intermediate (CD4^{int}) and high (CD4^{hi}) staining intensity (FIG. 3a). At four days, CD4 intensity remained Gaussian, similar to that evident at baseline and at six hours. By six days, a clear shoulder consisting of the CD4^{hi} population was evident; this population was well defined by seven days (FIG. 3a). This increase in surface intensity was not related to an increased size of a CD4⁺ blast population, as the increase in fluorescence intensity (as much as 10-fold) was disproportionate to cell size as assessed by scatter and not seen in similar analyses of other surface markers that would also be expected to increase in proportion to blast size (data not shown).

To determine the association between CD4 upregulation and the expression of activation markers on the cell surface, PBMC from healthy donors were stimulated with pools of allogeneic stimulator cells for seven days and then the intensity of CD4 expression and the expression of ten different T cell activation markers simultaneously assessed (FIG. 3b). Given known variances in the kinetics of activation marker upregulation, some markers were expressed more intensely following chronic activation using either viral antigens or alloantigens (e.g., CD38, CD58 and HLA-DR), while others (e.g., CD69 and CD103) were present at only minimally increased intensity on the surface of activated T cells. Regardless of the intensity of activation marker expression following allogeneic stimulation, increased expression of each activation marker occurred only on the subset of CD4+ T cells that upregulated surface CD4 following stimulation (i.e., CD4^{hi} T cells) (FIG. 3b). A close association between CD4 upregulation and activation marker expression was observed; for each marker assessed, CD4^{hi} cells were all activation marker positive, while CD4^{int} cells were activation marker negative. Similar results were seen when PBMC from CMV-seropositive donors were stimulated for seven days with CMV lysates.

### EXAMPLE 5

To determine whether CD4 upregulation was also associated with the proliferation of activated human CD4 T cells, healthy donor PBMC were labeled using the fluorescent dye CFSE prior to stimulation with either CMV or pools of alloantigenic PBMC. CFSE fluorescence decreases in intensity only in CD4^{hi} cells, suggesting that proliferation, similar to activation marker expression, was exclusively restricted to the CD4^{hi} subset (FIG. 3c). Conversely, CFSE^{low} cells did not occur within the CD4^{int} population, suggesting that these cells had not proliferated, consistent with the lack of activation marker expression on these cells following stimulation. Because CFSE fluorescence intensity declines linearly following cell division, the number of times that CD4 cells divided during the seven day stimulation period could not be calculated. Based on five experiments from five healthy donors, cells divided approximately four times during seven days (FIG. 3d). Based on the assumption that non-dividing cells did not preferentially die during the culture period, the starting frequency of alloantigen-specific T cells from the final frequencies of CD4^{hi}CD38+ T cells within the total CD4⁺ population was extrapolated. The median frequency of alloantigen specific CD4⁺ T cells was estimated at 6.9%±1.5%. This estimate is consistent with theoretical estimates of the frequency of alloantigen-specific cells in the T cell receptor repertoire, but significantly higher than prior estimates of alloreactive T cell frequencies obtained using limiting dilution assay methods.

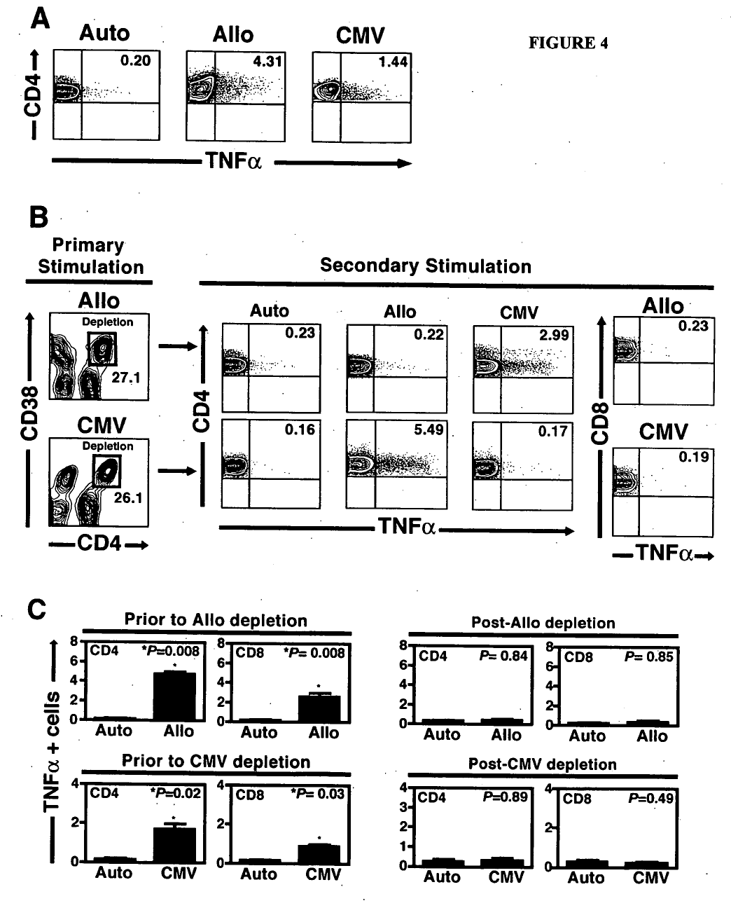

Elimination of specific CD4⁺ and CD8⁺ T cell function by depletion of activated CD4^{hi} T cells. PBMC from CMV-seropositive healthy donors was obtained and stimulated with either CMV lysates or irradiated pools of allogeneic stimulator cells. While CD4 upregulation could be used independently to define activation, cells were sorted based on the simultaneous expression of a secondary activation marker to define a sort gate that most clearly resolved subpopulations of activated CD4⁺ T cells. Based upon an analyses of the association between the expression of multiple activation markers and CD4 upregulation (FIG. 3b), the expression of CD38 was particularly well suited to two-dimensional discrimination of activation within CD4⁺ T cells. Following seven days of stimulation with a pool of allogeneic stimulator cells, the PBMC population was stained with antibodies to surface CD4 and CD38 and then high-speed sorting was used to deplete cells with the CD4^{hi}CD38⁺ phenotype. An example of a typical experiment is shown (FIG. 4). Few PBMC from a healthy CMV-seropositive donor produced intracellular TNFα following autologous stimulation (0.2%) while significant frequencies of CD4⁺ T cells responded to stimulation with pooled allogeneic stimulators or CMV lysates (4.31% and 1.44% of CD4⁺ T cells, respectively, FIG. 4a). Following seven days of stimulation with either pooled allogeneic PBMC or CMV lysates, CD4^{hi}CD38⁺ T cells were depleted by high-speed sorting (FIG. 4b). Following depletion, the residual PBMC were again stimulated with either the primary stimulus or a different secondary stimulus. CFC assays examining the production of TNFα within the CD4⁺ T cell population were carried out to assess secondary responses to CMV or alloantigen challenge using.

Following alloantigen stimulation and the depletion of CD4^{hi}CD38⁺ T cells, the residual population failed to respond to repeat alloantigen challenge above the baseline level of cytokine production seen with autologous stimulation (0.22% of CD4⁺ T cells vs. 0.23% for autologous re-stimulation, FIG. 4b). Importantly, CMV reactivity within the depleted CD4⁺ T cell population was preserved, and was slightly higher than the baseline frequency (2.99% of CD4⁺ T cells), as expected given the depletion of a CD4⁺ subpopulation devoid in CMV specificity (FIG. 4b).

The same experiment was run in the reverse direction, stimulating first with a CMV lysate and then re-stimulating the population depleted of CD4^{hi}CD38⁺ T cells with either autologous stimulators, the allogeneic PBMC pool or CMV antigens. CMV reactivity was eliminated following depletion (0.17% TNFα+T cells upon re-stimulation vs. 0.16% for the autologous control sample), but responses to alloantigens were preserved (5.49% of CD4⁺ T cells producing TNFα upon restimulation, FIG. 4b), demonstrating that this approach may be used to deplete cells of any specificity.

### EXAMPLE 6

Given the established role of CD4⁺ T cells in maintaining the number and function of CD8⁺ T cells, the impact of CD4 allodepletion alone on the function of residual, unsorted CD8⁺ T cells was examined. Surprisingly, CD8⁺ T cells within the residual PBMC population were incapable of activation, as assessed by intracellular TNFα expression following allogeneic stimulation approximating that in samples stimulated with autologous PBMC, despite the upregulation of activation markers including CD38 within these CD8⁺ T cells during primary exposure to pooled alloantigenic stimulators (0.23% TNFα+ T cells in the CD8⁺ population, FIG. 4b). Importantly, these CD8⁺ T cells could still respond appropriately when stimulated with CMV viral lysates, suggesting that the loss of alloreactivity was not due to global anergy of residual CD8⁺ T cells (data not shown). A similar loss of functional reactivity of CMV-specific CD8⁺ T cells was seen after depletion of CD4^{hi}CD38⁺ T cells following stimulation with CMV viral lysates (FIG. 4b). These data, in aggregate, confirm that alloreactive CD4⁺ T cells are the primary cells responding in the MLR and that the selective depletion of alloreactive CD4 T cell clones abrogates alloreactivity following secondary challenge in the residual, unsorted CD8⁺ T cell population.

Aggregate data from depletion of alloreactive CD4⁺ T cells from five donors, and depletion of CD4^{hi}CD38⁺ T cells following CMV lysate stimulation in four donors, confirmed these results (FIG. 4c). Following initial allogeneic stimulation and depletion of alloreactive CD4^{hi}CD38⁺ T cells, alloreactivity in CD4⁺ and CD8⁺ T cells upon restimulation was not significantly different that that following re-stimulation with autologous cells. Similarly, depletion of CMV-reactive CD4^{hi}CD38⁺ T cells resulted in the loss of functional CD4⁺ and CD8⁺ T cell reactivity to a level statistically indistinguishable from that seen in cells stimulated with autologous PBMC (FIG. 4c).

### EXAMPLE 7

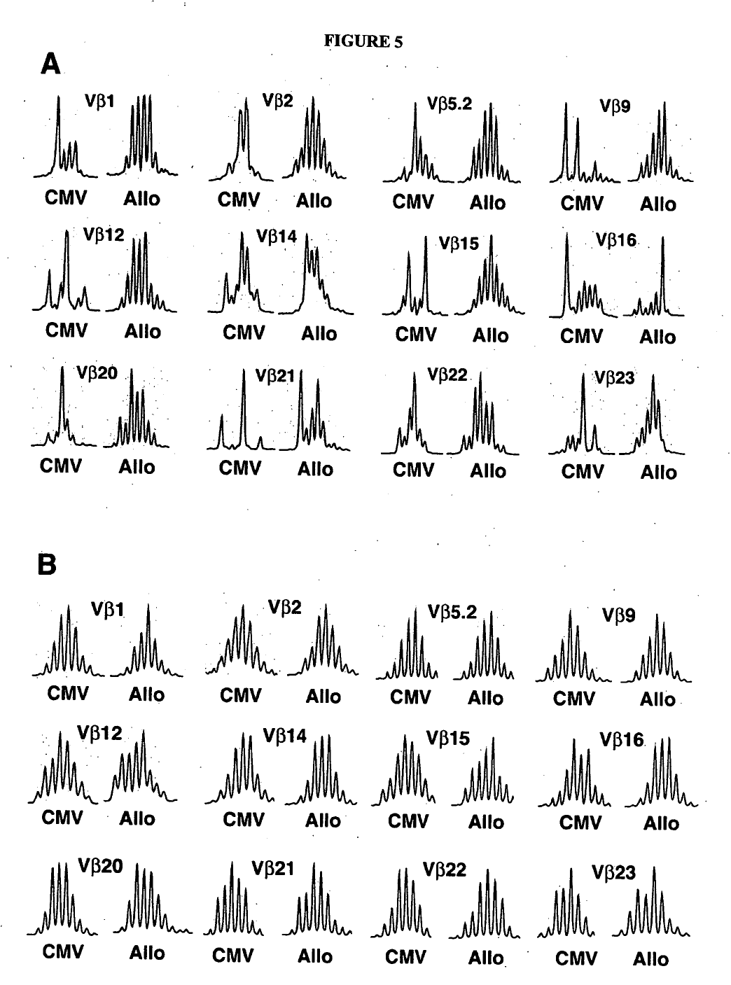

The residual T cell receptor repertoire following depletion of alloreactivity is diverse. The CDR3 regions in 23 TCR Vβ subsets was amplified by polymerase chain reaction (PCR) and the resulting PCR products subjected to electropheresis and quantitative densitometry to assess the fragment length diversity of the CDR3 region within each TCR Vβ family. Previously, spectratyping has been used to assess the overall diversity within the human TCR repertoire, while qualitative changes in TCR diversity by spectratyping have been used as a measure of immune reconstitution in humans. (Gorochov, et al., 1998) In multiple CMV-seropositive subjects, T cells were initially stimulated with both CMV lysates and pooled alloantigenic PBMC. Following each stimulus, populations of CD4^{hi}CD38⁺ and CD4^{int}CD38⁻ T cells were purified by cell sorting and analyzed the TCR Vβ diversity within each population by spectratyping (FIG. 5).

The CD4^{hi}CD38⁺ population obtained after CMV stimulation was the most restricted, with many TCR Vβ subsets demonstrating repertoire restriction consistent with one or several clones responding to CMV. In contrast, the CD4^{hi}CD38⁺ population isolated following alloantigen stimulation was more diverse, consistent with the large number of clones in the human TCR repertoire thought to be capable of responding to alloantigens (FIG. 5a). The CD4^{int}CD38⁻ T cell population, isolated following either CMV stimulation or stimulation using the alloantigen pool, was extremely diverse, as indicated by the Gaussian-appearing distribution of CDR3 fragment lengths within all TCR Vβ loci examined (FIG. 5b). These data demonstrate that following specific depletion of alloantigen-specific T cells, a T cell population not only capable of responding to a viral stimulus (e.g., CMV) but containing a broad TCR repertoire was preserved, reflecting the specificity of our depletion method.

### EXAMPLE 8

Depletion of alloreactive T cells following stimulation using cryopreserved apheresis products preserves viral and third-patty alloantigen responses. In a proposed embodiment, clinical application of alloreactive T cell depletion will require the use of cryopreserved PBMC products used as stimulators in one-way MLR reactions to deplete specific alloreactivity against a single HLA mismatched recipient.

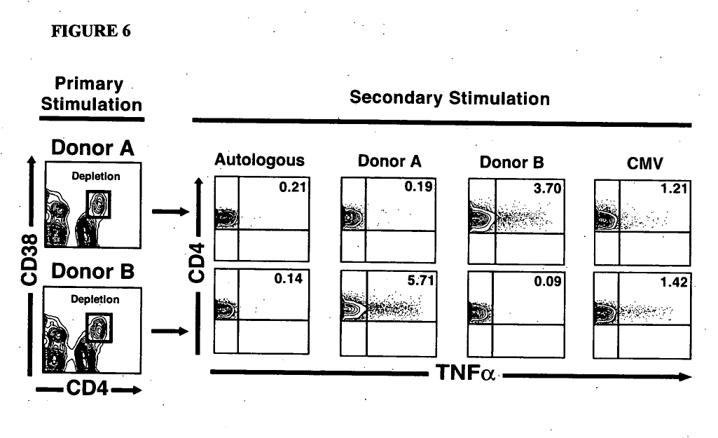

To determine the feasibility of this approach in a preclinical setting, residual cryopreserved apheresis products collected routinely in a clinical cellular processing laboratory were obtained. Products from two HLA-mismatched donors that were CMV seropositive (donors A and B) were used to stimulate cells from a healthy donor (donor C) in parallel one-way MLR stimulations (C→A and C→B). Following seven days of stimulation, CD4^{hi}CD38⁺ T cells were depleted by sorting and then re-stimulated residual cells with either the primary stimulus, autologous cells (C→A/C→C) and cells from the donor not used in the initial stimulation (C→A/C→B). Following depletion of alloantigen activated CD4^{hi}CD38⁺ T cells, repeat stimulation with the original donor resulted in no CD4⁺ T cell responsiveness above that seen following re-stimulation with autologous PBMC (0.19% for C→A/C→A vs. 0.21% for C→A/C→C; 0.09% C→B/C→B vs. 0.14% for C→B/C→C, FIG. 6).

Consistent with prior results, abrogation of alloreactivity within the non-depleted residual CD8⁺ T cell population occurred, with reduction of the alloresponses in this T cell subset to that seen in cell re-stimulated with autologous cells (data not shown). However, residual cells retained the ability to respond to a third-party stimulus as indicated by a significant frequency of responsive CD4⁺ T cells (3.70% for C→A/C→B and 5.71% for C→B/C→A, FIG. 6) and to CMV antigens (1.21% for C→A/C→CMV and 1.42% for C→B/C→CMV, FIG. 6). non-depleted CD8⁺ T cells also retained the capacity to respond to both alloantigens and to CMV antigens (data not shown). These data indicated that earlier methods, developed using pooled allogeneic PBMC as stimulators, were similarly effective when clinically obtained apheresis products were used instead.

Allogeneic stimulation. Stimulator and responder cells with desired degrees of HLA mismatching will be obtained either via thawing of previously cryopreserved or fresh apheresis products, or via ficoll sedimentation of PBMC obtained from heparinized peripheral blood specimens. Stimulator cells will be irradiated (at 25 Gy) and then co-incubated with responder cells at a 1:1 ratio at a cell concentration of 10⁶/ml in 2 ml final volume in a 24-well flat bottom plate. Cells will then be incubated in media (RPMI containing either 10% human AB serum or autologous responder serum) at 37° C. in 5% CO₂ for seven days. In all cases, parallel control stimulations will include media alone, irradiated autologous cells and cells stimulated with viral antigens (e.g., CMV lysate for stimulation of CMV-seropositive donors, who will comprise 60% of donor samples).

Assessment of alloreactivity using CFC. Following stimulation, irradiated stimulator cells are absent from the culture following approximately 48 hrs, as determined by labeling of stimulator cells using the highly fluorescent membrane dye PKH-2 (data not shown). Following the desired stimulation period (generally seven days), cells will be harvested, centrifuged and resuspended in RPMI+10% AB serum containing brefeldin A, an inhibitor of intracellular transport that allows cytokines to build up intracellularly. Following an additional 5 hrs, cells are washed (in PBS+2% AB serum) and resuspended in a cell permeabilization buffer (FACSLyse from Becton-Dickinson Immunocytometry Systems—BDIS). Cells will be washed again, and then stained with 3-4 fluorochrome-conjugated MAbs specific for surface markers and intracellular cytokines. A typical combination of MAbs might be CD4-FITC, TNFα-PE, CD14-PerCP and CD8-APC, to assess cytokine expression in CD4⁺ T cells (CD4⁺CD14⁻ TNFα+ cells) and CD8⁺ T cells (CD8⁺TNFα+ cells) within the lymphocyte gate by scatter. Data will be analyzed by flow cytometry, following acquisition of 100,000 events on a FACSCalibur or FACSAria cytometer.

### EXAMPLE 9

Depletion of alloreactive CD4+ T cells. Mixed-lymphocyte cultures will be established as outlined in the above Examples, using irradiated stimulator cells incubated with responder PBMC at a responder:stimulator ratio of 1:1 initially. Following seven days of stimulation, responder cells will be stained with fluorochrome-conjugated MAbs specific for CD4, CD38 and CD8. Initially, only cells with the CD4^{hi}CD38⁺ phenotype will be depleted. Remaining cells, including CD8⁺ cells (both CD38⁺ and CD38) and the residual CD4⁺ population (consisting of cells with intermediate levels of CD4 staining, or CD4^{int} cells) will be retained. Control stimulations will be performed using either no stimulus, viral antigens or irradiated autologous PBMC.

Assessment of residual antigen-specific T cell function and molecular diversity. Following sorting, residual cells will be rested (for 16 hrs in RPMI+10% AB or autologous serum at 37° C. in 5% CO₂). Following the rest period, they will be restimulated with either the primary allogeneic stimulus, cells from a third-party donor or viral lysates or peptide mixtures (e.g., derived from CMV or Epstein-Barr Virus (EBV) for virus-seropositive donors). It is expected that ˜60% of donors will be seropositive, while ˜90% will be EBV-seropositive. Following seven days of restimulation, cells will then be washed, resuspended in media containing brefeldin A (as in S.A. 1) and assessed for T cell function by examination of cytokine production (primarily TNFα, though IL-2, IFNγ and IL-4 will also be assessed in initial experiments) within gated populations of CD4⁺CD14⁻ and CD8⁺ lymphocytes (as described above). It is expected that control stimulations (e.g., with irradiated autologous cells) will result in −0.2% or fewer cytokine+ cells upon restimulation. Frequencies of CMV-specific or EBV-specific T cells within the CD4⁺ or CD8⁺ populations are expected to be in the 0.8-4% range. The diversity of the residual T cell repertoire will also be assessed by spectratyping. At the time of sort-based depletion, residual CD8⁺ T cells, CD4^{int}CD38⁻ and CD4^{hi}CD38⁺ T cells will be sorted separately (˜2×10⁶ each are expected to be required for spectratyping. Total RNA will be extracted using a commercial kit (for example, Tel-Test, Friendswood, Tex.) and prepared cDNA using reverse transcription (for example, Applied Biosystems, Foster City, Calif.). The CDR3 regions in 23 TCR Vβ subsets will be amplified by polymerase chain reaction (PCR) and the resulting PCR products subjected to capillary electrophoresis and quantitative densitometry to assess the fragment length diversity within each of the TCR Vβ families, as previously described. (Gorochov, et al., 1998; Kochenderfer, et al., 2002; Arstila, et al. 1999).

Depletion of alloreactive T cells in haploidentical and matched donor-recipient pairs. Sort-based depletion strategy will be assessed in three groups of donor-recipient pairs varying by relatedness: 1) Haploidentical donors and recipients; 2) Sibling donor-recipient pairs matched at ⅚ HLA loci; and 6) Matched sibling donors and recipients. Multiple donor-recipient pairs will be analyzed in each category; in each case, donor cells will be stimulated with irradiated recipient cells, media alone, and irradiated autologous PBMC. In each case, CD4^{hi}CD38⁺ T cells will be depleted following seven days of stimulation as described above, and residual cells will be rested and analyzed for residual alloreactivity, viral-specific T cell function (specific for CMV in CMV-seropositive donors or EBV in others) and for third-party alloreactivity.

Preclinical optimization and scale-up. Initial experiments utilized 24-well plates with 10⁶ each responder and stimulator cells, and then T25 flasks with 10⁷ cells each in a 20 ml volume. Scale-up experiments will initially use fully mismatched donor-recipient pairs (until the experiments of this aim demonstrate that our depletion approach works equally effectively in the setting of haploidentical or more closely matched pairs). T25 flasks with 10⁷ each responder and stimulator cells in a 1:1 ratio in 20 ml total volume will be used. The ratio of responders and stimulators in ratios varying from 1:1 to 20:1, will be used to determine the ratio at which depletion becomes less efficient. Subsequently, further scale-up cell numbers and volume using T75 flasks and, ultimately, closed bag systems will be incorporated. A final transition to the clinical lab will involve moving from the FACSAria cell sorter (BDIS) used to date in preclinical experiments to a more appropriate sortation device, such as, for example aclosed path cell sorter for express use in sorting cells for clinical purposes.

### EXAMPLE 10

While other patient groups will be targeted in subsequent trials, high-risk leukemia and lymphoma patients who lack a matched sibling or unrelated donor, who are unlikely to be cured without allogeneic SCT, and who have a suitable haploidentical donor will initially be target. These patients will include intermediate or high-grade lymphoma patients who fail autologous SCT, low-grade lymphoma patients who have failed at least two treatment regimens, Gleevec-unresponsive CML patients in accelerated phase or blast crisis, and high-risk patients with acute myeloid (AML) or lymphoid (ALL) leukemia (e.g., AML in 2nd or greater complete remission or with complex chromosomal abnormalities in 1st remission, secondary AML, or Philadelphia Chromosome-positive (Ph+) ALL patients in 1st remission).

Clinical allodepletion. Following G-CSF administration to donors, CD34+ stem cells will be collected following donor apheresis by routine clinical methods, to target an infusion dose of 5×10⁶/kg recipient weight. Additionally, 10⁸ mononuclear cells will be collected by apheresis of SCT donors (matched or mismatched with recipients as described above). Due to expected cell losses during irradiation (25 Gy), at least 3×10⁸ recipient PBMC will also be obtained by apheresis. Using sterile T75 flasks or a closed bag systems, responder and irradiated stimulator cells will be co-incubated for seven days at a ratio of 1:1 to 10:1 (using a safe ratio defined in Example 5). A parallel stimulation will also be performed using irradiated (25 Gy) autologous cells to establish background levels of activation. Following seven days of co-culture, cells will be stained with MAbs to CD4 and CD38 (conjugated to FITC and PE, respectively). In preliminary experiments, assuming an alloreactive precursor frequency of ˜5%, the CD4^{hi}CD38⁺ population has been found to comprise around 25% of total PBMC (due to 3-4 rounds of division among alloreactive T cells increasing their relative fraction of the overall pool). This number may be lower by up to five-fold in the setting of haploidentical or up to 10-fold in matched sibling recipients. Based on expected endpoint frequencies defined in Examples 4 and 5, sufficient cells will be sorted preferably using a closed path cell sorter for express use in sorting cells for clinical purposes under GMP conditions, with the population devoid of CD4^{hi}CD38⁺ T cells retained. This residual fraction will be tested for viability, loss of residual alloreactivity and retention of viral and third party alloantigen specificity (as described in S.A. 2) as well as for sterility and the presence of endotoxin and mycoplasma prior to product release. At least one log reduction in alloreactivity will initially be targeted for clinical release in the setting of haploidentical SCT, while 0.5 log reduction will be targeted in the matched sibling trial. While these targets serve as a minimal threshold, we expect that the degree of residual alloreactivity will be similar to that observed using control autologous stimulation based on experiments to date (as in FIG. 4).

Clinical trial design. The initial trial design will target haploidentical recipients receiving CD34-selected donor products supplemented with allodepleted T cells in a dose escalating phase I trial. The Miltenyi AC133 CliniMACS system or other available alternatives cell sorting will be used to obtain AC133+ cells (also enriched in CD34+ stem cells) from G-CSF mobilized donors. Haploidentical recipients will receive conditioning with Fludarabine (40 mg/m2) and Thymoglobulin (1.5 mg/kg)×4, (each administered daily days −6 through −3) and Thiotepa (10 mg/kg day −7). They will then receive at least 5×10⁶ CD34⁺ cells/kg recipient body weight combined with allodepleted T cells in a dose-escalating trial. Dose levels of allodepleted CD3⁺ T cells will include no allodepleted cells, 10⁴ allodepleted CD3⁺ cells/kg, 10⁵/kg, 10⁶/kg and 10⁷/kg recipient weight.

Statistical considerations. Stopping rules will be incorporated based on the development of adverse events, including GVHD, graft failure and infectious death, as each commonly causes morbidity and mortality after haploidentical SCT. Initially, at least 3 patients will be treated at dose level 0 (AC133+ stem cells only) with additional dose escalations consisting of 5-8 patients at each level, assuming no dose-limiting toxicities are reached.

### EXAMPLE 11

The following example outlines an approach wherein donor PBMC will be stimulated with cells obtained from recipients and the T cell subpopulation enriched for host-reactive T cells for use as a therapy for relapsed malignancy after allogeneic SCT.

Isolation of PBMC from allogeneic SCT donors. PBMC will be obtained via standard apheresis techniques from healthy donors of SCT recipients. Following PBMC harvesting by apheresis, viability and total mononuclear cell counts will be confirmed (e.g., by trypan blue staining or via automated counting). In some cases, PBMC products will be further purified to reduce platelet and red blood cell counts via ficoll gradient sedimentation.

Isolation of stimulator cells from SCT recipients. Timing of recipient cell collection. Two potential sources of stimulator cells will be obtained from SCT recipients. Prior to transplantation, recipients who lack evidence of significant numbers of malignant cells in peripheral blood may have a standard apheresis procedure performed to obtain PBMC. If apheresis is not possible, peripheral blood will be collected in heparinized tubes and PBMC will be obtained by standard ficoll gradient sedimentation. The second potential source of stimulator cells will be the peripheral blood or bone marrow following relapse. While peripheral blood is preferred due to the ease of collection, pelvic marrow aspiration could also be performed, especially when counts in the periphery are suppressed in association with malignant relapse. Regardless of whether pre-SCT or post-SCT relapse samples are obtained, it is estimated that optimal stimulation will require 10⁸-10⁹ cells. It is expected that pre-SCT PBMC will consistently provide a suitable allogeneic stimulus, but will need to be collected in advance at a time when the possibility of relapse is not certain. However, post-SCT relapse PBMC or marrow specimens are likely to be enriched for populations of cancer cells (e.g., leukemic blasts or lymphomatous cells) that may preferentially express cancer antigens and better stimulate the activation of cancer-specific donor T cells. The disadvantage of the latter cells is that they may less consistently express alloantigens that stimulate donor cells, relative to PBMC collected prior to SCT.

Post-collection procedures. PBMC or marrow cells obtained from SCT recipients would be counted and assessed for viability. PBMC would then be irradiated (at 25 Gy) and then used freshly or cryopreserved prior to use as a stimulator population for activation of donor-derived PBMC. When recipient cells are cryopreserved, as will be necessary for populations of cells obtained prior to SCT, cell numbers will be counted upon thawing. However, viable cell numbers will not be used, as cells will have been previously irradiated and will not be expected to retain viability. Cell counts will be adjusted prior to recipient-derived stimulator cells being combined with non-irradiated responder cells obtained from the respective SCT donor.

Stimulation of donor cells with recipient cells. Donor PBMC will be resuspended in media (e.g., RPMI media supplemented with Penicillin and streptomycin) to which serum (either 10% pooled human AB serum or donor-derived serum) has been added. While cell culture volumes may need to be optimized based on ongoing results, approximately 10⁶ xcells/ml has been proven to be effective. Optimal responder:stimulator cell ratios will also need to be re-established, though 1:1 ratios of viable responder cells:irradiated stimulator cells have proven effective in experiments to date.

Culture vessels and incubation conditions. Experiments to date have utilized sterile tissue culture flasks (75 cm² or 175 cm² flasks). However, clinical application may also utilize other containers (e.g., closed bag systems with volumes of up to 600 cc). While agitation is not required for adequate stimulation of responder cells, the addition of side-to-side or rolling agitation may further increase cell to cell contact and improve the quality of stimulation. Cell culture will be accomplished in traditional incubators at 37° C. in a humidified atmosphere containing 5% CO₂. Experiments to date have demonstrated that optimal allostimulation occurs following 5-7 days of culture, enabling optimal visualization of CD4 upregulation and two-dimensional discrimination of activated donor-reactive T cells using flow cytometry/sorting by gating on cells upregulating CD4 (“CD4^{hi}” cells) and co-expressing a second activation marker (e.g., CD38, CD58 or CD25). Prior experiments have shown that following 1-2 days of culture, previously irradiated stimulator cells are no longer present in the culture, leaving only responder PBMC.

Enrichment of alloreactive T cells by sorting. Following incubation for 7 days, PBMC are harvested are resuspended in phosphate buffered saline (PBS). Cells are then stained, using standard procedures, using fluorochrome-conjugated monoclonal antibodies (MAb) specific for CD4 and a secondary activation marker. Cells may also be counterstained with an antibody specific for CD14 to allow exclusion of monocytes (e.g., by gating on CD14-negative cells). Following staining, cells are washed and resuspended in PBS containing 1% human AB serum and disodium edetate (5 mM final concentration). Cells are then sorted using a high-speed flow sorter by sequentially gating on lymphocytes (by forward and side scatter), on CD14-negative cells (if CD14 staining is used) and on CD4^{hi} cells that co-express a secondary activation marker (e.g., CD4^{hi}CD58⁺ cells). Sufficient cells should be collected to facilitate planned infusions of enriched T cells. Cells exceeding the number of the initial infusion will be cryopreserved (in 90% AB serum and 10% dimethylsulfoxide—DMSO) for subsequent thawing and infusion. Following sorting, an aliquot of the enriched fraction should be retested for viability and to verify the degree of purification of the desired T cell subset. Cells will generally be collected in to PBS containing serum. Enriched cells will be centrifuged to reduce the volume and resuspended in the desired volume of sterile saline for injection into the recipient. Prior to testing, standard measurements will include sterility testing (e.g., bacterial cultures and tests to rule out mycoplasma contamination) and testing for endotoxin.

Selection of patients for therapy. SCT recipients who have relapsed will be considered for DLI if they have no evidence of active graft-versus-host disease (GVHD) following withdrawal of immunosuppression designed to prevent GVHD (e.g., cyclosporine A or tacrolimus and/or steroid therapy). In some cases, chemotherapy may be administered prior to the planned infusion of donor lymphocytes in order to reduce disease burden to facilitate the success of immunotherapy.

Dose escalation of DLI. Donor lymphocyte infusion may be accomplished by infusions of bulk lymphocytes or in the preferred mode of dose escalating infusions. Standard initial doses of infused lymphocytes during dose escalation may approximate 10⁷ cells/kg recipient body weight when DLI is accomplished using a sibling donor, or 10⁶ cells/kg recipient weight when an unrelated donor is used. Because it is expected that the potency of donor-specific enriched T cells will be significantly greater (≧1-2 logs), ˜10⁴ cells/kg will be used as an initial starting dose. Subsequent doses will be administered only if graft-versus-host disease is not observed and the relapsed malignancy is persistent. Subsequent doses would be given at least 1-2 weeks following the initial dose, and would be accomplished by infusing successively higher doses increased by 1 log (e.g., 10⁵ cells/kg for the second dose). The infusion of additional doses would be halted following any clinical evidence of graft-versus-host disease (e.g., manifested by a skin rash, elevations if liver function tests or gastrointestinal manifestations including diarrhea) or following documented disease regression.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the methods described herein without departing from the concept, spirit and scope of the invention. More specifically, it will be apparent that certain agents that are chemically or physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

Martins S., St John L., Champlin R., Wieder E., McMannis J., Molldrem J., Komanduri K., Functional assessment and specific depletion of alloreactive human T cells using flow cytometry, Blood;104(12):3429-36, (2004).

Bach, F. H., Bach, M. L. & Sondel, P. M. Differential function of major histocompatibility complex antigens in T-lymphocyte activation. Nature 259, 273-81 (1976).

Sprent, J., Schaefer, M., Lo, D. & Komgold, R. Properties of purified T cell subsets. II. In vivo responses to class I vs. class II H-2 differences. Journal of Experimental Medicine 163, 998-1011 (1986).

Ferrara, J. L. & Deeg, H. J. Graft-versus-host disease. New England Journal of Medicine 324, 667-74 (1991).

Ho, V. T. & Soiffer, R. J. The history and future of T-cell depletion as graft-versus host disease prophylaxis for allogeneic hematopoietic stem cell transplantation. Blood 98, 3192-204 (2001).

Champlin, R. et al. Allogeneic hematopoietic transplantation as adoptive immunotherapy. Induction of graft-versus-malignancy as primary therapy. Hematology-Oncology Clinics of North America 13, 1041-57 (1999).

Reisner, Y. et al. Transplantation for acute leukemia with HLA-A and B nonidentical parental marrow cells fractionated with soybean agglutinin and sheep red blood cells. Lancet 2, 327-31 (1981).

Prentice, H. G. et al. Use of anti-T-cell monoclonal antibody OKT3 to prevent acute graft-versus-host disease in allogeneic bone-marrow transplantation for acute leukaemia. Lancet 1, 700-3 (1982).

Filipovich, A. H. et al. Pretreatment of donor bone marrow with monoclonal antibody OKT3 for prevention of acute graft-versus-host disease in allogeneic histocompatible bone-marrow transplantation. Lancet 1, 1266-9 (1982).

Rodt, H. et al. Effect of anti-T-cell globulin on GVHD in leukemic patients treated with BMT. Transplantation Proceedings 13, 257-61 (1981).

Waldmann, H. et al. Elimination of graft-versus-host disease by in-vitro depletion of alloreactive lymphocytes with a monoclonal rat anti-human lymphocyte antibody (CAMPATH-1). Lancet 2, 483-6 (1984).

Cavazzana-Calvo M. et al. Improving immune reconstitution while preventing graft-versus-host disease in allogeneic stem cell transplantation. Seminars in Hematology 39, 32-40 (2002).

Goldman, J. M. et al. Bone marrow transplantation for chronic myelogenous leukemia in chronic phase. Increased risk for relapse associated with T-cell depletion. Annals of Internal Medicine 108, 806-14 (1988).

Couriel, D. et al. Early reactivation of cytomegalovirus and high risk of interstitial pneumonitis following T-depleted BMT for adults with hematological malignancies. Bone Marrow Transplantation 18, 347-53 (1996).

Arstila, T. P. et al. A direct estimate of the human alphabeta T cell receptor diversity. Science 286, 958-61 (1999).

Kochenderfer, J. N., Kobayashi, S., Wieder, E. D., Su, C. & Molldrem, J. J. Loss of T-lymphocyte clonal dominance in patients with myelodysplastic syndrome responsive to immunosuppression. Blood 100, 3639-45 (2002).

Waldrop, S. L., Pitcher, C. J., Peterson, D. M., Maino, V. C. & Picker, L. J. Determination of antigen-specific memory/effector CD4+ T cell frequencies by flow cytometry: evidence for a novel, antigen-specific homeostatic mechanism in HIV-associated immunodeficiency. Journal of Clinical Investigation 99, 1739-50 (1997).

Komanduri, K. V. et al. Restoration of cytomegalovirus-specific CD4+ T-lymphocyte responses after ganciclovir and highly active antiretroviral therapy in individuals infected with HIV-1. Nature Medicine 4, 953-6 (1998).

Lyons, A. B. Analysing cell division in vivo and in vitro using flow cytometric measurement of CFSE dye dilution. Journal of Immunological Methods 243, 147-54 (2000).

Orosz, C. G., Adams, P. W. & Ferguson, R. M. Frequency of human alloantigenreactive T lymphocytes. II. Method for limiting dilution analysis of alloantigenreactive helper T cells in human peripheral blood. Transplantation 43, 718-24 (1987).

Gorochov, G. et al. Perturbation of CD4+ and CD8+ T-cell repertoires during progression to AIDS and regulation of the CD4+ repertoire during antiviral therapy. Nature Medicine 4, 215-21 (1998).

Cavazzana-Calvo, M. et al. Specific elimination of alloreactive T cells by an anti-interleukin-2 receptor B chain-specific immunotoxin. Transplantation 50, 1-7 (1990).

Montagna, D. et al. Depletion of alloreactive T cells by a specific anti-interleukin-2 receptor p55 chain immunotoxin does not impair in vitro antileukemia and antiviral activity. Blood 93, 3550-7 (1999).

Stanzani, M. et al. CD25 expression on donor CD4+ or CD8+ T cells is associated with an increased risk of graft-versus-host disease following HLA-identical stem cell transplantation in humans. Blood 103, 1140-6 (2004).

Hoffmann, P., Ermann, J., Edinger, M., Fathman, C. G. & Strober, S. Donor-type CD4(+)CD25(+) regulatory T cells suppress lethal acute graft-versus-host disease after allogeneic bone marrow transplantation. Journal of Experimental Medicine 196, 389-99 (2002).

Matthias Edinger, P. H., Joerg Ermann, Kathryn Drago, C Garrison Fathman, Samuel Strober & Robert S Negrin. CD4+CD25+ regulatory T cells preserve graft-versus-tumor activity while inhibiting graft-versus-host disease after bone marrow transplantation. Nature Medicine 9, 1144-1150 (2003).

Godfrey, W. R., Krampf, M. R., Taylor, P. A. & Blazar, B. R. Ex vivo depletion of alloreactive cells based on CFSE dye dilution, activation antigen selection, and dendritic cell stimulation. Blood 103, 1158-65 (2004).

Peggs, K. S. et al. Adoptive cellular therapy for early cytomegalovirus infection after allogeneic stem-cell transplantation with virus-specific T-cell lines. Lancet 362, 1375-7 (2003).

Taylor, P. A., Lees, C. J. & Blazar, B. R. The infusion of ex vivo activated and expanded CD4(+)CD25(+) immune regulatory cells inhibits graft-versus-host disease lethality. Blood 99, 3493-9 (2002).

Janssen, E. M. et al. CD4+ T cells are required for secondary expansion and memory in CD8+ T lymphocytes. Nature 421, 852-6 (2003).

Molldrem, J. J. et al. Evidence that specific T lymphocytes may participate in the elimination of chronic myelogenous leukemia. Nature Medicine 6, 1018-23 (2000).

Emerson. S., “Ex Vivo Expansion of Hematopoietic Precursors, Progenitors, and Stem Cells: The Next Generation of Cellular Therapeutics”, Blood, 87:3082-88 (1996).

Baum, C. et al., “Isolation of a candidate human hematopoietic stem-cell population”, Proc. Natl. Acad. Sci. USA, 89:2804-8 (1992).

Lansdorp, P. M. et al., “Selective Expression of CD45 Isoforns on Functional Subpopulations of CD34.sup.+ Hemopoietic Cells from Human Bone Marrow”, J. Exp. Med., 172:363-66 (1990).

Petzer, A; et al., “Self-renewal of primitive human hematopoietic cells (long-term-culture-initiating cells) in vitro and their expansion in defined medium”, Proc. Natl. Acad. Sci. USA, 93:1470-74 (1996).

WO 95 11692, Davis et al.

Sato, N. et al., “Purification of Human Marrow Progenitor Cells and Demonstration of Direct Action of Macrophage Colony-Stimulating Factor on Colony-Forming Unit-Macrophage”, Blood, 78:967-74 (1991).

Smith, C. et al., “Purification and Partial Characterization of a Human Hematopoietic Precursor Population”, Blood 77:2122-28 (1991).

Udomsakdi, C. et al., “Characterization of Primitive Hematopoietic Cells in Normal Human Peripheral Blood”, Blood, 80:2513-21 (1992).

Udomsakdi, C. et al., “Separation of Functionally Distinct Subpopulation of Primitive Human Hematopoietic Cells using Rhodamine-123”, Exp. Hematol., 19:338-42 (1991).

Zandstra, P. et al., “Expansion of Hematopoietic Progenitor Cell Populations in Stirred Suspension Bioreactors of Normal Human Bone Marrow Cells”, Bio/Technology, 12:909-914 (1994).

Kochenderfer, J. N., Kobayashi, S., Wieder, E. D., Su, C., and Molldrem, J. J. 2002. Loss of T-lymphocyte clonal dominance in patients with myelodysplastic syndrome responsive to immunosuppression. Blood 100:3639-3645.

Arstila, T. P., Casrouge, A., Baron, V., Even, J., Kanellopoulos, J., and Kourilsky, P. 1999. A direct estimate of the human alphabeta T cell receptor diversity. Science 286:958-961.

Chen, B. J., Cui, X., Sempowski, G. D., Liu, C., and Chao, N. J. 2004. Transfer of allogeneic CD62L-memory T cells without graft-versus-host disease. Blood 103:1534-1541.

Anderson, B. E., McNiff, J., Yan, J., Doyle, H., Mamula, M., Shlomchik, M. J., and Shlomchik, W. D. 2003. Memory CD4⁺ T cells do not induce graft-versus-host disease. J Clin Invest 112:101-108.

Zhang, Y., Joe, G., Zhu, J., Carroll, R., Levine, B., Hexner, E., June, C., and Emerson, S. G. 2004. Dendritic Cell Activated CD44^{hi}CD8⁺ T Cells Are Defective in Mediating Acute Graft-versus-host Disease but Retain Graft-versus-leukemia Activity. Blood; 103: 3970-3978.

Goggins, T. and Chao, N., 2003. Depletion of host reactive T cells by photodynamic cell purging and prevention of graft versus host disease, Leuk Lymphoma ;44(11):1871-9

Immunochemical Methods In Cell And Molecular Biology (Mayer and Walker, eds., Academic Press, London, 1987)

Handbook Of Experimental Immunology, Volumes I-IV (D. M. Weir and C. C. Blackwell, eds., 1986)

Padley et al. 2001, Mature myeloid dendritic cells for clinical use prepared from CD14⁺ cells isolated by immunomagnetic adsorption, J. Hematother. Stem Cell Res. 10:427-429.

Dzionek et al. 2000, BDCA-2, BDCA-3, and BDCA-4: three markers for distinct subsets of dendritic cells in human peripheral blood, J. Immunol. 165: 6037-6046.

Berger T G, et al., 2005, “Efficient Elutriation of Monocytes Within a Closed System (Elutra™) for Clinical Scale Generation of Dendritic Cells.” Journal of Immunological Methods 298: 61-72.

Garlie N, et al., 2004, “Enrichment of CD 14+ Cells for Dendritic Cell Production Using the Elutra™ Cell Separation System.” Journal of Immunotherapy 27, no. 6: S22.

Noga S J, et al., 2001, “Development of a High Capacity Semi-Automated Monocyte Isolation System for the Generation of Dendritic Cells (DC).” Blood 98, no. 11, 1 of 2: 658a.

Read E J, et al., 2004, “Enrichment of Peripheral Blood Monocytes for Dendritic Cell Immunotherapy Using the Elutra® Cell Separation System.” Cytotherapy 6, no. 4:437.

Read E J, et al., 2003, “Evaluation of a Closed Automated System to Isolate Peripheral Blood Monocytes for Dendritic Cell (DC) Immunotherapy.” Cytotherapy 5, no. 5: 456.

Rouard H, et al., 2003, “A Closed and Single-Use System for Monocyte Enrichment: Potential for Dendritic Cell Generation for Clinical Applications.” Transfusion 43: 481-487.

Rouard H, et al., 2001, “Highly Purified Monocytes for Dendritic Cells Differentiation Using the Closed Single Use Cobe® Spectra™ CPS System.” Blood 98, no. 11, 1 of 2: 340b.

Adamson L, et al., 2004, “Development of a Technology Platform for Large-Scale Clinical Grade Production of DC.” Cytotherapy 6, no. 4: 363-371.

Cao H, et al., 2000, “In Vitro Generation of Dendritic Cells from Human Blood Monocytes in Experimental Conditions Compatible for In Vivo Cell Therapy.” Journal of Hematotherapy and Stem Cell Research 9: 183-194.

Faradji A, et al., 1994 “Large Scale Isolation of Human Blood Monocytes by Continuous Flow Centrifugation Leukapheresis and Counterflow Centrifugation Elutriation for Adoptive Cellular Immunotherapy in Cancer Patients.” Journal of Immunological Methods 174: 297-309.

Wong E C C, et al., 2001, “Development of a Clinical Scale Method for Generation of Dendritic Cells from PBMC for Use in Cancer Immunotherapy.” Cytotherapy 3, no. 1: 19-29.

Felzmann T, et al., 2003, “Monocyte Enrichment from Leukapheresis Products for the Generation of DCs by Plastic Adherence, or by Positive or Negative Selection.” Cytotherapy 5, no. 5: 391-398.

Svane I M, et al., 2003, “Clinical Application of Dendritic Cells in Cancer Vaccination Therapy.” APMIS 111: 818-834.

What is claimed is:

1. A method for generating a leukocyte population that is functionally anergic following restimulation with alloantigen, comprising:
a. stimulating a cell population with allogeneic stimulator cells; and
b. depleting T cells from said cell population expressing CD4^{hi} and a second activation marker.

2. The method of claim 1, wherein said depletion is by flow cytometry.

3. The method of claim 1, wherein said second activation marker is CD38.

4. The method of claim 1, wherein said second activation marker is CD25.

5. The method of claim 1, wherein said second activation marker is CD58.

6. The method of claim 1, wherein said cell population retains T cells capable of responding to antigens other than those expressed by said allogeneic stimulator cells.

7. The method of claim 1, wherein said allogeneic stimulator cells comprise dendritic cells.

8. A method of reducing the risk of graft versus host disease in a transplant recipient, comprising:
a. isolating a blood product from a donor;
b. stimulating cells in said blood product with allogeneic stimulator cells;
c. depleting cells from said blood product expressing CD4^{hi} and a second activation marker; and
d. administering said blood product to a recipient.

9. The method of claim 8, wherein said depletion is by flow cytometry.

10. The method of claim 8, wherein said second activation marker is CD38.

11. The method of claim 8, wherein said second activation marker is CD25.

12. The method of claim 8, wherein said second activation marker is CD58.

13. The method of claim 8, wherein said blood product retains T cells capable of responding to antigens other than those expressed by said allogeneic stimulator cells.

14. The method of claim 8, wherein said allogeneic stimulator cells comprise dendritic cells.

15. A cellular composition suitable for administration to a transplant recipient, wherein said cellular composition is produced by a process comprising:
a. isolating blood product from a donor;
b. stimulating cells in said blood product with allogeneic stimulator cells;
c. depleting cells from said blood product expressing CD4^{hi} and a second activation marker to form a cellular composition substantially free of cells expressing CD4^{hi} and said second activation marker.

16. The cellular composition of claim 15, wherein said depletion is by flow cytometry.

17. The cellular composition of claim 15, wherein said second activation marker is CD38.

18. The cellular composition of claim 15, wherein said second activation marker is CD25.

19. The cellular composition of claim 15, wherein said second activation marker is CD58.

20. The cellular composition of claim 15, wherein said cellular composition retains T cells capable of responding to antigens other than those expressed by said allogeneic stimulator cells.

21. The cellular composition of claim 15, wherein said allogeneic stimulator cells comprise dendritic cells.

22. A leukocyte population, wherein said leukocyte population is depleted of cells expressing CD4^{hi} and a second activation marker following stimulation with alloantigenic stimulator cells.

23. The leukocyte population of claim 22, wherein said second activation marker is CD38.

24. The leukocyte population of claim 22, wherein said second activation marker is CD25.

25. The leukocyte population of claim 22, wherein said second activation marker is CD58.

26. The leukocyte population of claim 22, wherein said cells are depleted by flow cytometry.

27. The leukocyte population of claim 22, wherein said leukocyte population retains T cells capable of responding to antigens other than those expressed by said allogeneic stimulator cells.

28. The leukocyte population of claim 22, wherein said allogeneic stimulator cells comprise dendritic cells.

29. A method for treating a patient with a hematopoietic cell cancer comprising administering to said patient purified T cells expressing CD4^{hi} and a second activation marker following stimulation with allogeneic stimulator cells.

30. The method of claim 29, wherein said second activation marker is CD38.

31. The method of claim 29, wherein said second activation marker is CD25.

32. The method of claim 29, wherein said second activation marker is CD58.

33. The method of claim 29, wherein said purified T cells are isolated by flow cytometry.

34. The method of claim 29, wherein said allogeneic stimulator cells are dendritic cells.

35. A method of reducing the risk of graft versus host disease in a transplant recipient, comprising:
a. isolating apheresis product from a donor;
b. stimulating cells in said apheresis product with allogeneic stimulator cells;
c. depleting cells expressing a CD4^{hi}CD38⁺ from said cellular composition by flow cytometry to form a cellular composition substantially free of CD4^{hi}CD38⁺ cells; and
d. administering said cellular composition to a recipient.

36. The method of claim 35, wherein said cellular composition is enriched for pathogen specific T cells prior to administration to said recipient.

37. The method of claim 35, wherein said cellular composition is administered to said recipient at a concentration of about 1×10⁴ cells/kg.

38. The method of claim 35, wherein said cellular composition is administered to said recipient at a concentration of about 1×10⁵ cells/kg.

39. The method of claim 35, wherein said cellular composition is administered to said recipient at a concentration of about 1×10⁶ cells/kg.

40. The method of claim 35, wherein said cellular composition is administered to said recipient at a concentration of about 1×10⁷ cells/kg.

41. The method of claim 35, wherein said cellular composition is administered to said recipient at a concentration of about 1×10⁸ cells/kg.

42. The method of claim 35, wherein said allogeneic stimulator cells are dendritic cells.

43. A method for reducing the autoimmune T-cells in a patient, comprising:
a. isolating apheresis product from said patient;
b. stimulating cells in said apheresis product with autoantigen;
c. depleting cells expressing a CD4^{hi}CD38⁺ from said cellular composition by flow cytometry to form a cellular composition substantially free of CD4^{hi}CD38⁺ cells; and
d. administering said cellular composition to said patient.

44. A method for generating a leukocyte population that is functionally anergic following restimulation with alloantigen, comprising:
a. stimulating a cell population with allogeneic dendritic cells; and
b. depleting T cells from said cell population expressing CD4^{hi} and a second activation marker.

45. The method of claim 44, wherein said depletion is by flow cytometry.

46. The method of claim 44, wherein said second activation marker is CD38.

47. The method of claim 44, wherein said second activation marker is CD25.

48. The method of claim 44, wherein said second activation marker is CD58.

49. The method of claim 44, wherein said cell population retains T cells capable of responding to antigens other than those expressed by said allogeneic dendritic cells.

50. A method for generating a leukocyte population that is functionally enriched following stimulation with a target antigen, comprising:
a stimulating a cell population with stimulator cells presenting the target antigen; and
b. selecting T cells from said cell population expressing CD4^{hi} and a second activation marker.

51. The method of claim 50, wherein said selection is by flow cytometry.

52. The method of claim 50, wherein said second activation marker is CD38.

53. The method of claim 50, wherein said second activation marker is CD25.

54. The method of claim 50, wherein said second activation marker is CD58.

55. The method of claim 50, wherein said stimulator cells comprise dendritic cells.

56. The method of claim 50, wherein said target antigen is a tumor antigen.

57. The method of claim 50, wherein said target antigen is a pathogen antigen.

58. The method of claim 50, wherein said target antigen is a viral antigen.

59. A cellular composition, wherein said cellular composition is produced by a process comprising:
a. isolating blood product from a donor;
b. stimulating cells in said blood product with stimulator cells presenting a target antigen;
c. selecting cells from said blood product expressing CD4^{hi} and a second activation marker to form a cellular composition enriched with cells expressing CD4^{hi} and said second activation marker.

60. The cellular composition of claim 59, wherein said selection is by flow cytometry.

61. The cellular composition of claim 59, wherein said second activation marker is CD38.

62. The cellular composition of claim 59, wherein said second activation marker is CD25.

63. The cellular composition of claim 59, wherein said second activation marker is CD58.

64. The cellular composition of claim 59, wherein said stimulator cells comprise dendritic cells.

65. The cellular composition of claim 59, wherein said target antigen is a tumor antigen.

66. The cellular composition of claim 59, wherein said target antigen is a pathogen antigen.

67. The cellular composition of claim 59, wherein said target antigen is a viral antigen.
